(19) United States
(12) Patent Application Publication
(10) Pub. No.: US 2003/0180947 A1
(43) Pub. Date: Sep. 25, 2003

(54) Circadian control of stem/progenitor cell self-renewal and differentiation and of clock controlled gene expression

(76) Inventors: J.H. David Wu, Pittsford, NY (US); Yi-Guang Chen, Rochester, NY (US); Athanassios Mantalaris, Middlesex (GB); Matthew Heckman, Rochester, NY (US)

Correspondence Address: Michael L. Goldman, NIXON PEABODY LLP, Clinton Square, P.O. Box 31051, Rochester, NY, 14603, US

(21) Appl. No.: 10/252,990

(22) Filed: Sep. 23, 2002

Related U.S. Application Data
(60) Provisional application No. 60/324,190, filed on Sep. 21, 2001.

(51) Int. Cl. A61K 48/00; C12N 5/08; C12N 15/85

(52) U.S. Cl. 435/455; 435/372; 514/44

(57) ABSTRACT

Methods of controlling bone marrow cell development, stem cell self-renewal, differentiation and/or function, and expression of clock controlled genes having an E-box sequence in their regulatory region by providing appropriate cells having a circadian clock system and manipulating the circadian clock system under conditions effective to control bone marrow cell development, stem cell self-renewal, differentiation and/or function, as well as expression of clock controlled genes having an E-box sequence in their regulatory region. In addition, an in vitro engineered tissue is disclosed that includes a plurality of cells or cell types in intimate contact with one another to form a tissue, the cells or cell types having a circadian clock system that has been modulated to regulate growth, development and/or functions of the cells or cell types within the tissue.

This application claims the priority benefit of U.S. Provisional Patent Application Serial No. 60/324,190 filed Sep. 21, 2001, which is hereby incorporated by reference in its entirety.

The present invention was made, at least in part, with funding received from the National Science Foundation, Grant No. BES-9631670, and the National Aeronautics and Space Administration, Grant No. NAG 8-1382. The U.S. government may have certain rights in this invention.

### FIELD OF THE INVENTION

The present invention relates generally to the use of circadian control systems for in vitro development of stem cells and engineered tissues, in vivo modification of stem cells and tissue development, and in vitro and in vivo control over clock controlled gene expression.

### BACKGROUND OF THE INVENTION

The molecular components of the mammalian clock system have been recently identified (Albrecht et al., “A differential response of two putative mammalian circadian regulators, mper1 and mper2, to light,” Cell 91:1055-1064 (1997); Honma et al., “Circadian oscillation of BMAL1, a partner of a mammalian clock gene Clock, in rat suprachiasmatic nucleus,” Biochem. Biophys. Res. Commun. 250:83-87 (1998); Kume et al., “mCRY1 and mCRY2 are essential components of the negative limb of the circadian clock feedback loop,” Cell 98:193-205 (1999); Sangoram et al., “Mammalian circadian autoregulatory loop: a timeless ortholog and mPer1 interact and negatively regulate CLOCK-BMAL1-induced transcription,” Neuron 21:1101-1113 (1998); Sun et al., “RIGUI, a putative mammalian ortholog of the Drosophilaperiod gene,” Cell 90:1003-1011 (1997); Tei et al., “Circadian oscillation of a mammalian homologue of the Drosophila period gene,” Nature 389:512-516 (1997); Zylka et al., “Three period homologs in mammals: differential light responses in the suprachiasmatic circadian clock and oscillating transcripts outside of brain,” Neuron 20:1103-1110 (1998)). They consist of positive regulators, CLOCK and BMAL1, and negative regulators, PER1, PER2, PER3, TIM, CRY1 and CRY2. In the clock system, the expression of the period genes is controlled by a feedback mechanism (Dunlap, “Molecular bases for circadian clocks,” Cell 96:271-290 (1999)). As a result of this feedback control, the expression of the period genes oscillates in a circadian manner. Circadian oscillation of the clock genes has been reported in suprachiasmatic nucleus (“SCN”), where the central pacemaker is located. The clock genes have also been found to be expressed and oscillate in several peripheral tissues (Zylka et al., “Three period homologs in mammals: differential light responses in the suprachiasmatic circadian clock and oscillating transcripts outside of brain,” Neuron 20:1103-1110 (1998); Sakamoto et al., “Multitissue circadian expression of rat period homolog (rPer2) mRNA is governed by the mammalian circadian clock, the suprachiasmatic nucleus in the brain,” J. Biol. Chem. 273:27039-27042 (1998); Balsalobre et al., “A serum shock induces circadian gene expression in mammalian tissue culture cells,” Cell 93:929-937 (1998)), including liver, skeletal muscle and testis, which indicates the existence of the circadian clock in at least some of the peripheral tissues.

The circadian rhythms of different aspects of hematopoiesis have been documented in both human and murine systems (Laerum, “Hematopoiesis occurs in rhythms,” Exp. Hematol. 23:1145-1147 (1995); Smaaland, “Circadian rhythm of cell division,” Prog. Cell. Cycle. Res. 2:241-266 (1996)). In the studies involving mice (Levi et al., “Circadian and seasonal rhythms in murine bone marrow colony-forming cells affect tolerance for the anticancer agent 4′-O-tetrahydropyranyladriamycin (THP),” Exp. Hematol. 16:696-701 (1988); Perpoint et al., “In vitro chronopharmacology of recombinant mouse IL-3, mouse GM-CSF, and human G-CSF on murine myeloid progenitor cells,” Exp. Hematol. 23:362-368 (1995); Wood et al., “Distinct circadian time structures characterize myeloid and erythroid progenitor and multipotential cell clonogenicity as well as marrow precursor proliferation dynamics,” Exp. Hematol. 26:523-533 (1998); Aardal and Laerum, “Circadian variations in mouse bone marrow,” Exp. Hematol. 11:792-801 (1983); Aardal, “Circannual variations of circadian periodicity in murine colony-forming cells,” Exp. Hematol. 12:61-67 (1984)), the numbers of colony-forming units (“CFUs”) in bone marrow, including the multipotent colonies (CFU-GEMM), burst-forming unit-erythrocyte (BFU-E), CFU-erythrocyte (CFU-E) and CFU-granulocyte, macrophage (CFU-GM) have been shown to be circadian dependent. Furthermore, erythroid and myeloid lineages showed distinct and different circadian rhythms confirmed by CFU assays and cell cycle analysis (Wood et al., “Distinct circadian time structures characterize myeloid and erythroid progenitor and multipotential cell clonogenicity as well as marrow precursor proliferation dynamics,” Exp. Hematol. 26:523-533 (1998)). Similarly, in human studies (Smaaland et al., “DNA synthesis in human bone marrow is circadian stage dependent,” Blood 77:2603-2611 (1991); Abrahamsen et al., “Variation in cell yield and proliferative activity of positive selected human CD34+ bone marrow cells along the circadian time scale,” Eur. J. Haematol. 60:7-15 (1998); Smaaland et al., “Colony-forming unit-granulocyte-macrophage and DNA synthesis of human bone marrow are circadian stage-dependent and show covariation,” Blood 79:2281-2287 (1992); Abrahamsen et al., “Circadian cell cycle variations of erythro- and myelopoiesis in humans,” Eur. J. Haematol. 58:333-345 (1997)), significant circadian variations in the DNA synthesis activity were observed in both myelopoiesis and erythropoiesis (Abrahamsen et al., “Circadian cell cycle variations of erythro- and myelopoiesis in humans,” Eur. J. Haematol. 58:333-345 (1997)). The number of CFU-GM shows a significant 24-hour rhythm and correlated with the DNA synthesis activity in the bone marrow cells (Smaaland et al., “Colony-forming unit-granulocyte-macrophage and DNA synthesis of human bone marrow are circadian stage-dependent and show covariation,” Blood 79:2281-2287 (1992)). Despite these well-documented observations, the molecular events controlling the circadian variations remain elusive.

It has been demonstrated that immortalized SCN cell lines, such as SCN2.2 cells, possess the capacity to generate circadian rhythms endogenously and, like SCN cells in vivo, to confer this rhythmicity to other cells via a diffusible signal (Allen et al., “Oscillating on borrowed time: diffusible signals from immortalized suprachiasmatic nucleus cells regulate circadian rhythmicity in cultured fibroblasts,” J. Neurosci. 21(20):7937-43 (2001)).

A number of clock controlled genes (CCGs) have also been identified. These include, for example, vasopressin (Jin et al., “A molecular mechanism regulating rhythmic output from the suprachiasmatic circadian clock,” Cell 96:57-68 (1999)); serotonin N-acetyltransferase (Chong et al., “Characterization of the chicken serotonin N-acetyltransferase gene activation via clock gene heterodimer/E box interaction,” J. Biol. Chem. 275:32991-32998 (2000)); arylalkylamine N-acetyltransferase (Chen and Baler, “The rat arylalkylamine N-acetyltransferase E-box: differential use in a master vs. a slave oscillator,” Mol. Brain Res. 81:43-50 (2000)); and Prokineticin 2 (Cheng et al., “Prokineticin 2 transmits the behavioural circadian rhythm of the suprachiasmatic nucleus,” Nature 417(6887):405-410 (2002)). However, none of these CCGs has been shown to be regulated in bone marrow tissues.

The questions of whether bone marrow contains its own clock system and whether the known clock elements (and therefore CCGs) are expressed in bone marrow have not been explored. Therefore, it would be desirable to identify whether bone marrow is indeed under control of a circadian clock system and, if so, to identify also the molecular components of its circadian clock system and uses thereof.

The present invention is directed to overcoming these and other deficiencies in the art.

### SUMMARY OF THE INVENTION

One aspect of the present invention relates to a method of controlling bone marrow cell development that includes: providing bone marrow cells having a circadian clock system and manipulating the circadian clock system under conditions effective to control bone marrow cell development.

Another aspect of the present invention relates to a method of controlling stem cell self-renewal, differentiation and/or functions, said method including: providing stem cells having a circadian clock system and manipulating the circadian clock system under conditions effective to control stem cell self-renewal, differentiation and/or functions.

A further aspect of the present invention relates to an in vitro engineered tissue that includes: a plurality of cells or cell types in intimate contact with one another to form a tissue, the cells or cell types having a circadian clock system that has been modulated to regulate growth, development, and/or functions of the cells or cell types within the tissue.

Still further aspects of the present invention relate to methods of controlling expression of a clock controlled gene that includes: providing a cell having a circadian clock system and manipulating the circadian clock system of the cell under conditions effective to alter expression of a clock controlled gene selected from the group consisting of GATA Binding Protein (GATA)-2, interleukin (IL)-12, IL-16, granulocyte-macrophage-colony stimulating factor (GM-CSF)-2, LATS2, Bone Morphogenetic Protein (BMP)-2, BMP-4, Telomerase Reverse Transcriptase (catalytic subunit) (TERT), Transforming Growth Factor (TGF)-β1, TGF-β2, TGF-β4, Piwi-like-1, CCAAT/enhancer binding protein (C/EBP)-α, Dentin Matrix Protein (DMP)-1, Old Astrocyte Specifically Induced Substance (OASIS), LIM homeobox protein (Lhx)-2, Homeo Box B4 (hox-B4), Paired Box Gene 5 (Pax5), and Cilliary Neurotrophic Factor Receptor (CNTFR). By controlling expression of the various clock controlled genes, it is possible to (i) treat diseases or enhance or modify body functions or activities (e.g., jet lag, shift work) mediated by expression or deficiency of a particular clock controlled gene; and (ii) enhance the immune system and/or influence cell self-renewal, proliferation, differentiation, activity, longevity, function, and/or potency.

The present invention relates to the identification of molecular control mechanisms that can be harnessed to control and manipulate the circadian clock system of cells in various tissues, thereby regulating the expression of various proteins involved in cell growth and differentiation and providing an approach for treating diseases or enhancing or modifying a body's functions or activities related to under- or over-expression of such proteins. One molecular control mechanism utilized in the circadian clock system for controlling the expression of various proteins regulated in circadian manner (i.e., the product of clock-controlled genes or CCGs) is the presence in the regulatory region of an element designated herein as an E-box (CANNTG, SEQ ID No: 1, where N is any nucleotide).

### BRIEF DESCRIPTION OF THE DRAWINGS

FIGS. 1A-B illustrate the expression of mPer1 in murine bone marrow cells. FIG. 1A shows a representative result of the relative quantitative RT-PCR analysis of the mPer1 expression at different circadian times; and FIG. 1B shows the relative amount of mPer1 mRNA at different Zeitgeber Time (ZT). The intensity of the DNA band corresponding to mPer1 was normalized to that of the 18S rRNA internal control. Within each experiment, the highest normalized level was set as 100% and the relative amount of mRNA was calculated. Each value represents the mean±SEM of the results from four to five mice (one way ANOVA, p<0.01). The horizontal bar at the bottom represents the light-dark cycle. Data at ZT 0 and 20 are plotted twice.

FIGS. 2A-B illustrate the expression of mPer2 in murine bone marrow cells. FIG. 2A shows a representative result of the relative quantitative RT-PCR analysis of the mPer2 expression at different circadian times; and FIG. 2B shows the relative amount of mPer2 mRNA at different Zeitgeber Time (ZT). The relative amount of mPer2 mRNA was calculated as described in the legend to FIG. 1. Each value represents the mean±SEM of the results from four to five mice (one way ANOVA, p=0.07). The horizontal bar at the bottom represents the light-dark cycle. Data at ZT 0 and 20 are plotted twice.

FIGS. 3A-B illustrate the expression of mPer1 and mPer2 in the myeloid enriched (Gr-1 positive) fraction of murine bone marrow cells. The relative amount of mPer mRNA was calculated as described in the legend to FIG. 1. FIG. 3A shows the relative amount of mPer1 mRNA at different Zeitgeber Times (ZT). FIG. 3B shows the relative amount of mPer2 mRNA at different Zeitgeber Time. The data in 3A and 3B represent the mean±SEM of the results from four to six mice. * p<0.05 as compared to the value at ZT 4. The horizontal bar at the bottom represents the light-dark cycle. Data at ZT 0 and 20 are plotted twice.

FIG. 4 illustrates schematically the identification and approximate location of three CACGTG (SEQ ID No: 2) E-boxes upstream of exon IS in mouse GATA-2 (SEQ ID No: 3). Two first exons are denoted as IS and IG. Three E-box elements are in bold. The Xho I site is underlined. The locations of six different inserts (3a-1, -2, -3, -4, -7, and -14) are indicated at the bottom. The original insert in the genomic DNA clone is composed of 3a-2 and 3a-4. E: EcoR I; N: Not I.

FIG. 5 illustrates the enhanced transcriptional activity of the IS promoter in the presence of CLOCK and BMAL1. Transcriptional activation of the luciferase reporter containing the wild-type IS promoter (pGL3-3a-7) or the truncated promoter (pGL3-3a-31 and pGL3-3a-39). The locations of the three E-boxes (E) are indicated. H1299 cells were transiently transfected with the reporter plasmid (pGL3-3a-7, pGL3-3a-31, or pGL3-3a-39) in the presence (black bars) or absence (white bars) of mCLOCK and hBMAL1. For each reporter construct, data are presented as fold induction with respect to the corresponding control (without mCLOCK and hBMAL1). Each value is the mean±SEM of three replicates.

FIGS. 6A-B illustrate the expression of the mGATA-2 IG transcript in total murine bone marrow cells. In FIG. 6A, a representative result of the relative quantitative RT-PCR analysis of the mGATA-2 IG transcript is shown. In FIG. 6B, the relative amounts of the mGATA-2 IG transcript at different circadian times is shown. The intensity of the DNA band corresponding to the IG transcript was normalized to that of the 18S rRNA internal control. Within each experiment, the highest normalized level was set as 100 and the relative amounts of mRNA were calculated. Each value represents the mean±SEM of the results from four replicates (one way ANOVA, p<0.05). The horizontal bar at the bottom represents the light-dark cycle. Data at 0 and 20 hours are plotted twice.

FIGS. 7A-B illustrate the expression of the mGATA-2 IS transcript in lin⁻ murine bone marrow cells. In FIG. 7A, a representative result of the relative quantitative RT-PCR analysis of the mGATA-2 IS transcript is shown. In FIG. 7B, the relative amounts of the mGATA-2 IS transcript at different circadian times is shown. The intensity of the DNA band corresponding to the IS transcript was normalized to that of the 18S rRNA internal control. Within each experiment, the highest normalized level was set as 100 and the relative amounts of mRNA were calculated. At each time point, the lin⁻ cells were obtained from the total bone marrow cells of two mice. Each value represents the mean±SEM of the results from three replicates (one way ANOVA, p<0.05). The horizontal bar at the bottom represents the light-dark cycle. Data at 0 and 20 hours are plotted twice.

FIG. 8 illustrates the effects that each E-box in the GATA-2 IS promoter region has in mediating CLOCK and BMAL1-dependent transactivation. A schematic diagram depicting constructs pGL3-E1b-GEs, -GE1, -GE2 and -GE3 is at the top. H1299 cells were transiently transfected with the luciferase reporter construct containing three or individual E-boxes (E) and their flanking regions. Presence (+) or absence (−) of the reporter and the expression plasmids is indicated. The results are presented as fold induction with respect to the control reporter vector (pGL3-E1b). Each value is the mean±SEM of three replicates.

FIG. 9 illustrates the negative regulation of CLOCK and BMAL1 transcriptional activity through the GATA-2 IS promoter by individual PER proteins. H1299 cells were transiently transfected with the reporter plasmid (pGL3-3a-7) in the presence (+) or absence (−) of the expression plasmids as denoted. Each value is the mean±SEM of three replicates. E: E-box.

FIGS. 10A-C illustrate the nucleotide and protein sequences as well as overall structure of mlats2b and mlats2c . FIG. 10A shows the nucleotide and protein sequences of mlats2b (SEQ ID Nos: 4 and 5). FIG. 10B shows the nucleotide and protein sequences of mlats2c (SEQ ID Nos: 6 and 7). The stop codon is indicated by an asterisk. The start codon is assigned according to the mLATS2 sequence (GenBank Accession BAA92380, which is hereby incorporated by reference in its entirety). The putative splicing site is indicated by a short arrow. The putative polyadenylation signal is boxed. The numbers denote the positions of the first nucleotides or last amino acids of each line. The Pst I restriction site is underlined. FIG. 10C illustrates the general structure of mLATS2b and mLATS2c relative to mLATS2. The numbers denote the amino acid positions. The N-terminal 113 amino acids (black box) are identical for all three proteins. The insertion of 49 amino acids in mLATS2c is shown by an open box. The meshed box indicates the identical region between mLATS2b and mLATS2c. FIG. 10C is not drawn to scale.

FIG. 11 illustrates the expression of mlats2, mlats2b, and mlats2c in murine bone marrow. RT-PCR was performed in the presence (+) or absence (−) of reverse transcriptase to analyze mlats2, mlats2b and mlats2c expression in murine bone marrow. The PCR products of mlats2 (483 bp), mlats2b (379 bp) and mlats2c (525 bp) are indicated by arrowheads.

FIGS. 12A-B illustrate the circadian expression profiles of mlats2 and mlats2b in total bone marrow cells. In FIG. 12A, the relative amounts of mlats2 mRNA are shown at different times. * p<0.05 as compared to the values at 4 hours after light onset (t test). In FIG. 12B, the relative amounts of mlats2b mRNA are shown at different times. * p<0.05 as compared to the values at 4 and 20 hours after light onset (t test). The intensity of the DNA band corresponding to mlats2 or mlats2b was normalized to that of the 18S rRNA internal control. Within each experiment, the highest normalized level was set as 100 and the relative amounts of mRNA at other time points were calculated. Each value represents the mean±SEM of the results from three mice. The horizontal bar at the bottom represents the light-dark cycle. Data at 0 and 20 hours after light onset are plotted twice.

FIG. 13 shows an alignment and comparison of the mouse and human LATS2 proteins. The top panel shows the high homology within the N-terminal regions and the kinase domains as indicated by the percentages of identity in amino acid sequences. The numbers denote the amino acid positions. The horizontal bar indicates the approximate size of 100 amino acids. The bottom panel shows the sequence alignment of the N-terminal regions (mouse LATS2, SEQ ID No: 8; human LATS2, SEQ ID No: 9). The GenBank Accessions are BAA92380 for mLATS2 (which is hereby incorporated by reference in its entirety) and AAF80561 for hLATS2/KPM (which is hereby incorporated by reference in its entirety). Identical residues are shown by shaded background. A gap is indicated by a dash.

FIG. 14 is a bar graph illustrating the effects of neurotransmitter analog treatment on NIH 3T3 cells transfected with pGL3-mPer1-7.2kb, which contains luciferase under control of a 7.2 kb region of the mper1 promoter. Cells were exposed to 10⁻⁶ M forskolin as a positive control, 10⁻⁶ M isoproterenol (a beta-adrenergic agonist), 10⁻⁶ M propranolol (a beta-adrenergic antagonist), 10⁻⁶ M phenylephrine (an alpha-adrenergic agonist), and 10⁻⁶ M pentolamine (an alpha-adrenergic antagonist) for 7 hours.

### DETAILED DESCRIPTION OF THE INVENTION

The present invention relates to the identification of molecular control mechanisms that can be harnessed to control and manipulate the circadian clock system of cells in various tissues, thereby regulating the expression of various proteins involved in cell growth and differentiation and providing an approach for treating diseases or enhancing or modifying body functions or activities related to under- or over-expression of such proteins. The molecular control mechanism utilized in the circadian clock system for controlling the expression of various proteins regulated in circadian manner (i.e., the product of clock-controlled genes or CCGs) is the presence in their upstream or other regulatory regions of an element designated herein as an E-box.

It appears that the transcriptional regulation of CCGs is an important means by which the circadian clock carries out its function. A clock-controlled gene can be directly regulated by the clock components (e.g., CLOCK and BMAL1). If a clock-controlled gene encodes a transcription factor, rhythmic accumulation of this transcription factor may direct circadian expression of its downstream genes. As a result, the circadian clock can control many genes simultaneously.

The E-box is a nucleic acid sequence as follows: CANNTG (SEQ ID No: 1) where N can be any nucleotide. It is believed that all CCGs in various tissues are characterized by the presence of one or more E-boxes in their upstream or other regulatory regions. Having identified the presence of the E-box in a number of different CCGs and having demonstrated that positive and negative regulators can influence the expression levels of CCGs, particularly in bone marrow tissue, the present invention affords a method of controlling expression of CCGs and, thus, controlling certain phenotypic changes that involve expression of those CCGs.

As used herein, “circadian clock system” is used to convey the meaning that cells, either in vivo or in vitro, are provided with a complete or partial complement of positive and negative regulators of the circadian clock (as needed). It is now known that the positive regulators are CLOCK and BMAL1 while the negative regulators are PER1, PER2, PER3, TIM, CRY1 and CRY2. These regulators are also called clock elements.

A number of signaling molecules are known to regulate or modulate the activity of positive or negative regulators of the circadian clock system. For example, it is now known that signal molecule(s) produced by suprachiasmatic nucleus (SCN) and glucocorticoids modulate the clock elements. As disclosed herein, it has also been discovered that some neurotransmitters or their analogs have the capability of modulating the clock elements. As used herein, signaling molecules can be any of the above-described molecules or other signaling molecules that later become identified.

Thus, modulation of the circadian clock system of target cells can be carried out by exposing the target cells to the signaling molecule(s) of SCN cells or exposing the target cells to glucocorticoids or neurotransmitters (as well as analogs thereof) that can modulate the clock elements. Additional approaches for modulation of the circadian clock system include, without limitation, transfecting a target cell with either a constitutive or an inducible engineered gene that encodes one or more clock elements or signaling molecules; introducing into the target cell an RNA molecule or a protein (e.g., fusion protein), where the RNA encodes or the fusion protein contains a clock element or signaling molecule (or active fragment thereof). Still further approaches for modulating the circadian clock system of target cells involves modifying the redox potential in the environment where the target cells are located, i.e., via control of NADH levels, control of oxygen levels, or control consumption rate with carbonyl cyanide m-chlorophenylhydrazone (Rutter et al., “Regulation of clock and NPAS2 DNA binding by the redox state of NAD cofactors,” Science 293:510-514 (2001); Takahashi et al., “Mitochondrial respiratory control can compensate for intracellular O₂ gradients in cardiomyocytes at low PO₂,” Am. J. Physiol. Heart Circ. Physiol. 283(3):H871-878 (2002), each of which is hereby incorporated by reference in its entirety) or adding lactate to culture media; changing an individual's feeding scheme to modulate the circadian clock system in certain tissues (e.g., liver) (see Rutter et al., “Metabolism and the control of circadian rhythms,” Annu. Rev. Biochem. 71:307-331 (2002), which is hereby incorporated by reference in its entirety), and changing an individual's exposure to light and dark cycles. Other approaches for modulating the circadian clock system, whether previously or subsequently developed, can also be employed in the present invention.

The target cells whose circadian clock system can be modulated in accordance with the present invention can be located in vivo, i.e., in a target tissue or organ, or in vitro, i.e., in a cell culture or engineered tissue.

Many in vivo tissues naturally contain a circadian clock system that can be manipulated by controlling the levels of the positive or negative regulators for purposes of regulating the expression of clock control genes (CCGs) that are under circadian control. Examples of tissue systems that are known to possess tissue-specific circadian control systems include, without limitation: liver, pancreas, skeletal muscle, testis, bone marrow, and heart. To modulate the circadian clock system of certain target cells in vivo, specific signaling molecules or positive or negative regulators can be administered to an individual (e.g., as a fusion protein) or RNA can be administered to an individual for uptake by target cells. Alternatively, gene therapy approaches (i.e., with either constitutive or inducible expression) can be performed. Finally, feeding schemes or light/dark exposure cycles can be modified to override the circadian clock system in target cells (or tissues).

For in vitro systems, one approach for modulating the circadian clock system of cultured target cells is to incubate the cultured cells with SCN cell lines that are known to express the various circadian clock genes and transmit circadian signals. The SCN cell lines are preferably in the same medium but not physically contacting the target cells (i.e., separated by a permeable membrane). Suitable SCN cell lines include SCN2.2 obtained by immortalizing primary fetal murine SCN cells (see Earnest et al., “Establishment and characterization of denoviral E1A immortalized cell lines derived from the rat suprachiasmatic nucleus,” J. Neurobiol. 39(1):1-13 (1999); Allen et al., “Oscillating on borrowed time: diffusible signals from immortalized suprachiasmatic nucleus cells regulate circadian rhythmicity in cultured fibroblasts,” J. Neurosci. 21(20):7937-43 (2001), each of which is hereby incorporated by reference in its entirety). The SCN cells will provide the cell culture with the circadian signals according to their normal circadian oscillation patterns. Alternatively, the positive and negative regulators can be introduced into cells in vitro. This can be achieved in a number of ways including, without limitation, protein or RNA transduction or recombinant expression of gene constructs using known recombinant technology.

In vitro Systems

The nucleic acid sequences of the circadian regulators is known: CLOCK (see GenBank Accession NM_{—}152221 (human) and NW 000231 (mouse), each of which is hereby incorporated by reference in its entirety), BMAL1 (see GenBank Accession NM_{—}001178 (human) and NW_{—}000332 (mouse), each of which is hereby incorporated by reference in its entirety); PER1 (see GenBank Accession NM_{—}002616 (human) and AF223952 (mouse), each of which is hereby incorporated by reference in its entirety); PER2 (see GenBank Accession NM_{—}022817 and NM_{—}003894 (human) and NM_{—}011066 (mouse), each of which is hereby incorporated by reference in its entirety); PER3 (see GenBank Accession NM_{—}016831 (human) and XM_{—}124453 (mouse), each of which is hereby incorporated by reference in its entirety); TIM (see GenBank Accession NT_{—}007933, NT_{—}007914, and NT_{—}004873 (human) and XM_{—}138545 (mouse), each of which is hereby incorporated by reference in its entirety); CRY1 (see GenBank Accession NM_{—}004075 (human) and NM_{—}007771 (mouse), each of which is hereby incorporated by reference in its entirety); and CRY2 (see GenBank Accession XM_{—}051030 (human) and XM_{—}130307 (mouse), each of which is hereby incorporated by reference in its entirety).

DNA molecules encoding the above-identified positive and negative regulators can be obtained using conventional molecular genetic manipulation for subcloning gene fragments, such as described by Sambrook et al., Molecular Cloning: A Laboratory Manual, Cold Springs Laboratory, Cold Springs Harbor, N.Y. (1989), and Ausubel et al. (ed.), Current Protocols in Molecular Biology, John Wiley & Sons (New York, N.Y.) (1999 and preceding editions), each of which is hereby incorporated by reference in its entirety. In conjunction therewith, DNA molecules can be obtained using the PCR technique together with specific sets of primers chosen to represent the upstream and downstream tennini of the open reading frames. Erlich et al., Science 252:1643-51 (1991), which is hereby incorporated by reference in its entirety.

Once the desired DNA molecule has been obtained, DNA constructs can be assembled by ligating together the DNA molecule encoding the open reading frames with appropriate regulatory sequences including, without limitation, a promoter sequence operably connected 5′ to the DNA molecule, a 3′ regulatory sequence operably connected 3′ of the DNA molecule, as well as any enhancer elements, suppressor elements, etc. The DNA construct can then be inserted into an appropriate expression vector. Thereafter, the vector can be used to transform a host cell and the recombinant host cell can express the positive or negative regulator.

For purposes of producing RNA transcripts or positive or negative regulators (i.e., as a fusion protein, non-fusion protein, or active fragment thereof) that can be administered to an individual, prokaryotic host cells are preferable. When a prokaryotic host cell is selected for subsequent transformation, the promoter region and polyadenylation region used to form the DNA construct (i.e., transgene) should be appropriate for the particular host. A number of suitable promoters (both constitutive and inducible), initiators, enhancer elements, and polyadenylation signals that are specific for prokaryotic expression are known in the art. For a review on maximizing gene expression, see Roberts and Lauer, Methods in Enzymology, 68:473 (1979), which is hereby incorporated by reference in its entirety.

Alternatively, eukaryotic cells, preferably mammalian cells, can also be used for purposes of producing RNA transcripts or positive or negative regulators. Suitable mammalian host cells include, without limitation: COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), CHOP, and NS-1 cells. A number of suitable promoters (both constitutive and inducible), initiators, enhancer elements, and polyadenylation signals that are specific for eukaryotic (more specifically, mammalian) expression are known in the art.

Regardless of the selection of host cell, once the desired DNA has been ligated to its appropriate regulatory regions using well known molecular cloning techniques, it can then be introduced into a suitable vector or otherwise introduced directly into a host cell using transformation protocols well known in the art (Sambrook et al., Molecular Cloning: A Laboratory Manual, Second Edition, Cold Spring Harbor Press, NY (1989), which is hereby incorporated by reference in its entirety).

The recombinant DNA construct can be introduced into host cells via transformation, particularly transduction, conjugation, mobilization, electroporation, or other suitable techniques. Suitable hosts include, but are not limited to, bacteria, yeast, mammalian cells, insect cells, plant cells, and the like. The hosts, when grown in an appropriate medium, are capable of expressing the RNA or positive or negative regulator or signaling molecule, which can then be isolated therefrom and, if necessary, purified. The RNA or positive and/or negative regulators or signaling molecules are preferably produced in purified form (preferably at least about 80%, more preferably 90%, pure) by conventional techniques, including immuno-purification techniques for protein recovery or hybridization protocols for RNA recovery.

The in vitro culturing of cells in accordance with the methods of the present invention can be carried out using a three-dimensional cell culture device or bioreactor that mimics the natural extracellular matrix and ample surface area, allowing cell to cell interaction at a tissue-like cell density that occurs in native tissues. It is understood that the bioreactor can have many different configurations so long as it provides a three-dimensional structure. Bioreactors of this type have been described in detail in U.S. patent application Ser. No. 09/715,852 to Wu et al., filed Nov. 17, 2000, and Ser. No. 09/796,830 to Wu et al., filed Mar. 1, 2001, each of which is hereby incorporated by reference in its entirety.

Basically, the bioreactor includes a container or vessel having within its confines a scaffolding upon which the various cells therein may grow and a suitable culture medium appropriate for the cells grown therein.

The walls of the container or vessel may comprise any number of materials such as glass, ceramic, plastic, polycarbonate, vinyl, polyvinyl chloride (PVC), metal, etc.

The scaffolding may consist of tangled fibers, porous particles, or a sponge or sponge-like material. Suitable scaffolding substrates may be prepared using a wide variety of materials including, without limitation, natural polymers such as polysaccharides and fibrous proteins; synthetic polymers such as polyamides (nylon), polyesters, polyurethanes; semi-synthetic materials; minerals including ceramics and metals; coral; gelatin; polyacrylamide; cotton; glass fiber; carrageenans; and dextrans. Exemplary tangled fibers include, without limitation, glass wool, steel wool, and wire or fibrous mesh. Examples of porous particles include, without limitation, beads (glass, plastic, or the like), cellulose, agar, hydroxyapatite, treated or untreated bone, collagen, and gels such as Sephacryl, Sephadex, Sepharose, agarose or polyacrylamide. “Treated” bone may be subjected to different chemicals such as, acid or alkali solutions. Such treatment alters the porosity of bone. If desired, the substrate may be coated with an extracellular matrix or matrices, such as, collagen, matrigel, fibronectin, heparin sulfate, hyaluronic and chondroitin sulfate, laminin, hemonectin, or proteoglycans.

The scaffolding essentially has a porous structure, with the pore size being determined by the cell types intended to occupy the bioreactor. One of skill in the art can determine the appropriate pore size and obtain suitable scaffolding materials that can achieve the desired pore size. Generally, a pore size in the range of from about 15 microns to about 1000 microns can be used. Preferably, a pore size in the range of from about 100 microns to about 300 microns is used.

In addition, the bioreactor can also contain a membrane to facilitate gas exchange. The membrane is gas permeable and may have a thickness in the range of from about 10 to about 100 μm, preferably about 40 to about 60 μm. The membrane is placed over an opening in the bottom or side of the chamber or container. In order to prevent excessive leakage of media and cells from the bioreactor, a gasket may be placed around the opening and/or a solid plate placed under or alongside the opening and the assembly fastened.

Culture media is placed over or around the porous or fibrous substrate. Suitable culture media need to support the growth and differentiation of cells of various tissues and (optionally) any accessory cells included therein. Exemplary culture media include, without limitation, (i) classical media such as Fisher's medium (Gibco), Basal Media Eagle (BME), Dulbecco's Modified Eagle Media (D-MEM), Iscoves's Modified Dulbecco's Media, Minimum Essential Media (MEM), McCoy's 5A Media, and RPMI Media, optionally supplemented with vitamin and amino acid solutions, serum, and/or antibiotics; (ii) specialized media such as MyeloCult™ (Stem Cell Technologies) and Opti-Cell™ (ICN Biomedicals) or serum free media such as StemSpan SFEM™ (StemCell Technologies), StemPro 34 SFM (Life Technologies), and Marrow-Gro (Quality Biological Inc.). A preferred media for bone marrow includes McCoy's 5A medium (Gibco) used at about 70% v/v, supplemented with approximately 1×10⁻⁶ M hydrocortisone, approximately 50 μg/ml penicillin, approximately 50 mg/ml streptomycin, approximately 0.2 mM L-glutamine, approximately 0.45% sodium bicarbonate, approximately 1×MEM sodium pyruvate, approximately 1×MEM vitamin solution, approximately 0.4×MEM amino acid solution, approximately 12.5% (v/v) heat inactivated horse serum and approximately 12.5% heat inactivated FBS, or autologous serum.

The culture medium can also be supplemented with signaling molecules of the type described above that can regulate or modify the expression of CCGs and/or clock elements.

In vivo Therapies

To augment the expression levels of positive or negative regulators in particular tissues or cells, protein-based delivery systems can be administered, nucleic acid delivery systems can be administered, or in vitro transfected cells can be administered. Regardless of the particular method of the present invention which is practiced, when it is desirable to manipulate the circadian clock system of a cell (i.e., to be treated) either positive or negative regulators can be taken-up by the cell or expressed therein.

One approach for delivering proteins or polypeptides or RNA molecules into cells involves the use of liposomes. Basically, this involves providing a liposome which includes that protein or polypeptide or RNA to be delivered, and then contacting the target cell with the liposome under conditions effective for delivery of the protein or polypeptide or RNA into the cell.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), which is hereby incorporated by reference). When liposomes are endocytosed by a target cell, for example, they can be routed to acidic endosomes which will destabilize the liposome and result in drug release.

This liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., each of which is hereby incorporated by reference in its entirety.

Yet another approach for delivery of proteins or polypeptides involves preparation of chimeric proteins according to U.S. Pat. No. 5,817,789 to Heartlein et al., which is hereby incorporated by reference in its entirety. The chimeric protein can include a ligand domain and, e.g., positive or negative regulator or other signaling molecule. The ligand domain is specific for receptors located on a target cell. Thus, when the chimeric protein is delivered intravenously or otherwise introduced into a target organ site, the chimeric protein will adsorb to the targeted cells and the targeted cells will internalize the chimeric protein. A number of approaches can be used, including adjuvants such as Bioporter, a lipid based transfection reagent (available from Gene Therapy Systems), Chariot (available from Active Motif; see Morris et al., “A peptide carrier for the delivery of biologically active proteins into mammalian cells,” Nature Biotech. 19:1173-1176 (2001), which is hereby incorporated by reference in its entirety), Pro-Ject, a cationic lipid based transfection reagent (available from Pierce), and TAT mediated fusion proteins (see Becker-Hapak et al., “TAT-mediated protein transduction into mammalian cells,” Methods 24:247-256 (2001), which is hereby incorporated by reference in its entirety).

When it is desirable to achieve heterologous expression of a particular protein or polypeptide or RNA molecule in a target cell, DNA molecules encoding the desired protein or polypeptide or RNA can be delivered into the cell. Basically, this includes providing a nucleic acid molecule encoding the RNA or positive or negative regulator or signaling molecule (described above) and then introducing the nucleic acid molecule into the cell under conditions effective to express the RNA or positive or negative regulator or signaling molecule in the cell. Preferably, this is achieved by inserting the nucleic acid molecule into an expression vector before it is introduced into the cell.

When transforming mammalian cells for heterologous expression of a protein or polypeptide, an adenovirus vector can be employed. Adenovirus gene delivery vehicles can be readily prepared and utilized given the disclosure provided in Berkner, Biotechniques 6:616-627 (1988) and Rosenfeld et al., Science 252:431-434 (1991), WO 93/07283, WO 93/06223, and WO 93/07282, each of which is hereby incorporated by reference in it entirety. Adeno-associated viral gene delivery vehicles can also be constructed and used to deliver a gene to cells. In vivo use of these vehicles is described in Flotte et al., Proc. Nat'l Acad. Sci. 90:10613-10617 (1993); and Kaplitt et al., Nature Genet. 8:148-153 (1994), each of which is hereby incorporated by reference in its entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; and U.S. Pat. No. 5,885,808 to Spooner et al.; and U.S. Pat. No. 5,871,727 to Curiel, each of which is hereby incorporated by reference in its entirety).

Retroviral vectors which have been modified to form infective transformation systems can also be used to deliver nucleic acid encoding a desired positive or negative regulator into a target cell. One such type of retroviral vector is disclosed in U.S. Pat. No. 5,849,586 to Kriegler et al., which is hereby incorporated by reference in its entirety.

Regardless of the type of infective transformation system employed, it should be targeted for delivery of the nucleic acid to a specific cell type. The infected cells will then express the desired RNA or positive or negative regulator or signaling molecule to modify the circadian clock system.

Alternatively, in vitro transfected cells can be administered to an individual. For example, bone marrow cells can be transfected to modulate their circadian clock system, cultured in a bioreactor of the type described above, and then administered to an individual, where the bone marrow cells take up residence in the individual's bone marrow. Similar approaches can be utilized for other tissues.

Utilities

As demonstrated in the Examples, bone marrow cells are directly regulated by the circadian clock system and, specifically, a number of CCGs are expressed in bone marrow cells under circadian control. One aspect of the present invention relates to controlling bone marrow cell development, either in vivo or in vitro. This aspect of the present invention can be carried out by providing bone marrow cells having a circadian clock system and then manipulating the circadian clock system under conditions effective to control bone marrow cell development.

The bone marrow cells whose development can be modified include, without limitation, stem cells (e.g., totipotent stem cells, pluripotent stem cells, myeloid stem cells, mesenchymal stem cells, and lymphoid stem cells); bone marrow progenitor cells (e.g., CFU-GEMM cells, Pre B cells, lymphoid progenitors, prothymocytes, BFU-E cells, CFU-Meg cells, CFU-GM cells, CFU-G cells, CFU-M cells, CFU-E cells, and CFU-Eo cells); bone marrow precursor cells (e.g., promonocytes, megakaryoblasts, myeloblasts, monoblasts, normoblasts, myeloblasts, proerythroblasts, B-lymphocyte precursors, and T-lymphocytes precursors); and cells with specific functions (e.g., natural killer (NK) cells, dendritic cells, bone cells including osteoclasts and osteoblasts, tooth cells such as odontoblasts and odontocytes, B-lymphocytes, T-lymphocytes, and macrophages). As a result of such modification, the affected cells can be directed to self-renew, enhance or modify function or activity, or develop into certain class of mature blood or bone marrow cells (e.g., megakaryocytes, neutrophilic myelocytes, eosinophilic myelocytes, basophilic myelocytes, erythrocytes, thrombocytes, polymorphonucleated neutrophils, monocytes, eosinophils, basophils, B-lymphocytes, T-lymphocytes, macrophages, mast cells, NK cells, dendritic cells, bone cells, and plasma cells) as well as other blood cells, liver cells, neural cells, muscle cells, chondrocytes, cartilage cells, bone cells including osteoclasts and osteoblasts, tooth cells including odontoblasts and odontocytes, fat cells, hematopoietic support cells, pancreatic cells, cornea cells, retinal cells, and heart muscle cells.

The bone marrow cells can be manipulated either to activate bone marrow cell development or, alternatively, to deactivate bone marrow cell development.

A related aspect of the invention concerns a method of controlling stem cell self-renewal, differentiation and/or functions, either in vivo or in vitro. This method is carried out by providing stem cells having a circadian clock system and then manipulating the circadian clock system under conditions effective to control stem cell self-renewal, differentiation and/or functions. Stem cells that can be treated include, without limitation, totipotent stem cells, pluripotent stem cells, myeloid stem cells, mesenchymal stem cells, neural stem cells, liver stem cells, muscle stem cells, fat tissue stem cells, skin stem cells, limbal stem cells, hematopoietic stem cells, AGM (aorta-gonad-mesonephros) stem cells, yolk sac stem cells, bone marrow stem cells, embryonic stem cells, embryonic germ cells, and lymphoid stem cells.

As a result of stimulating such differentiation, the stem cells can be directed to develop into liver cells, neural cells, muscle cells, chondrocytes, cartilage cells, bone cells, tooth cells, fat cells, hematopoietic support cells, pancreatic cells, cornea cells, retinal cells, or heart muscle cells.

Yet another aspect of the present invention relates to controlling the expression of various CCGs that contain E-boxes in their regulatory regions. Exemplary protein whose genes contain E-boxes and whose expression can therefore be controlled by manipulating the circadian clock system include, without limitation, GATA-2 (GenBank Accession NM_{—}002050, which is hereby incorporated by reference in its entirety), GM-CSF (GenBank Accession AJ224148, which is hereby incorporated by reference in its entirety), IL-12 (GenBank Accession U89323, which is hereby incorporated by reference in its entirety), IL-16 (GenBank Accession AF077011, which is hereby incorporated by reference in its entirety), LATS-2 and variants thereof (GenBank Accession NM_{—}014572, which is hereby incorporated by reference in its entirety), BMP-2 (see gi|20559789:6481126-6891400 Homo sapiens chromosome 20 reference genomic contig, which is available through GenBank and is hereby incorporated by reference), BMP-4 (see gi|20874093:c1747657-1642415 Mus musculus WGS supercontig Mm14_WIFeb10_{—}273 and gi|22048717:c35056312-34329142 Homo sapiens contig, each of which is available through GenBank and is hereby incorporated by reference in its entirety), TERT (see gi|18560952:1-92564 Homo sapiens contig and gi|20909147|ref|NW_{—}000084.1|Mm13_WIFeb01_{—}265 Mus musculus WGS supercontig Mm13_WIFeb01_{—}265, each of which is available through GenBank and is hereby incorporated by reference in its entirety), TGF-β1 (see gi|18590119:c1040201-951525 Homo sapiens contig and gi|20822543:1775929-1843023 Mus musculus WGS supercontig Mm7_WIFeb01_{—}149, each of which is available through GenBank and is hereby incorporated by reference in its entirety), TGF-β2 (see gi|20835056:c3324644-3050222 Mus musculus WGS supercontig Mm1_WIFeb01_{—}22, which is available through GenBank and is hereby incorporated by reference in its entirety), TGF-β3 (see gi|20909979:c31249210-30994966 Mus musculus WGS supercontig Mm12_WIFeb01_{—}235, which is available through GenBank and is hereby incorporated by reference in its entirety), Piwi-like-1 (see gi|18601829:814574-1034194 Homo sapiens contig, which is available through GenBank and is hereby incorporated by reference in its entirety), C/EBP-α (see gi|20826395:1538637-1613557 Mus musculus WGS supercontig Mm7_WIFeb01_{—}157, which is available through GenBank and is hereby incorporated by reference in its entirety), DMP-1 (see gi|20839315:8361795-8573110 Mus musculus WGS supercontig Mm5_WIFeb01_{—}80, which is available through GenBank and is hereby incorporated by reference in its entirety), OASIS (see gi|20841149:33045239-33303239 Mus musculus WGS supercontig Mm2_WIFeb01_{—}27, which is available through GenBank and is hereby incorporated by reference in its entirety), Lhx-2 (see gi|17449540:c4011934-3862220 Homo sapiens contig, which is available through GenBank and is hereby incorporated by reference in its entirety), hox-B4 (see gi|17480533:1-609558 Homo sapiens contig, which is available through GenBank and is hereby incorporated by reference in its entirety), Pax5 (see gi|17451799:c2761059-2564015 Homo sapiens contig, which is available through GenBank and is hereby incorporated by reference in its entirety), and CNTFR (see gi|17451799:25577-74425 Homo sapiens contig, which is available through GenBank and is hereby incorporated by reference in its entirety)

Regardless of the CCG whose protein expression levels are manipulated in cells, either in vitro or in vivo, this method of the present invention can be carried out by providing cells having a circadian clock system and then manipulating the circadian clock system of the cells under conditions effective to control expression of those CCGs. The cells that are treated can be any of the above-described stem cells, hematopoietic and/or stromal cells such as bone marrow progenitor cells and bone marrow precursor cells, and in certain circumstances mature blood or bone marrow cells. As a result of such treatment, expression levels of the targeted CCGs can be either deactivated or activated, depending on the positive or negative regulators or signaling molecules employed.

Thus, in accordance with this aspect of the invention, GATA-2 expression levels can be upregulated (activated) or downregulated (deactivated), thereby influencing stem cell self-renewal or differentiation.

Likewise, in accordance with this aspect of the invention, GM-CSF expression levels can be upregulated (activated) or downregulated (deactivated), thereby influencing hematopoietic and/or stromal cell and/or stem cell self-renewal or differentiation. Moreover, GM-CSF expression levels can be used to treat diseases mediated by GM-CSF or its deficiency such as type I neurofibromatosis, juvenile myelomonocytic leukemia, or myeloproliferative disorder. In addition, GM-CSF can be used to enhance the immune system and/or influence cell differentiation and/or potency as in the clearance of Group B streptococcus (see Online Mendelian Inheritance in Man (OMIM) 138960, which is hereby incorporated by reference in its entirety).

Further CCGs include one or more interleukins, such as IL-12 and IL-16. Thus, in accordance with this aspect of the present invention, IL-12 or IL-16 expression levels can be upregulated (activated) or downregulated (deactivated), thereby influencing hematopoietic and/or stromal cell and/or stem cell self-renewal or differentiation. Moreover, IL-12 and IL-16 can be used to enhance the immune system and/or influence cell differentiation and/or potency, and IL-12 may additionally be useful in preventing UV-induced skin cancer (see OMIM 161560 and 603035, each of which is hereby incorporated by reference in its entirety).

Yet another CCG whose expression levels can be controlled include LATS2, as well as splice variants thereof such as LATS2b and LATS2c. Thus, in accordance with this aspect of the present invention, expression levels LATS2 and its splice variants can be upregulated (activated) or downregulated (deactivated), thereby influencing hematopoietic and/or stromal cell and/or stem cell self-renewal or differentiation. Moreover, LATS2 (or its splice variants) expression levels can be used to treat diseases mediated thereby or its deficiency such as cancers, leukemias, or other proliferative or malignant diseases (see OMIM 604861, which is hereby incorporated by reference in its entirety).

Likewise, in accordance with this aspect of the invention, TERT expression levels can be upregulated (activated) or downregulated (deactivated), thereby influencing the replicative potential of hematopoietic and/or stromal cell and/or stem cells. Moreover, TERT expression levels can be used to treat diseases mediated by TERT such as the unlimited growth of cancers that is not checked by replicative senescence. In addition, TERT can be used to increase the replicative lifespan of cell lines in-vitro. See OMIM 187270, which is hereby incorporated by reference in its entirety.

Further CCGs include one or more bone morphogenesis proteins, such as BMP-2 and BMP-4. In accordance with this aspect of the present invention, BMP-2 and BMP-4 expression levels can be upregulated (activated) or downregulated (deactivated), thereby influencing bematopoietic and/or stromal cell and/or stem cell self-renewal or differentiation. In addition, BMP-2 and BMP-4 can be used to influence bone cell differentiation and development (see OMIM 112261 and 112262, each of which is hereby incorporated by reference in its entirety).

Additional CCGs include one or more growth factors, transcription factors, and differentiation inducing agents, such as TGF-β1, -β2 and -β3, Piwi-like-1, C/EBP-α, DMP-1, OASIS, Lhx-2, HoxB4, Pax5 and CNTFR. Thus, in accordance with this aspect of the present invention, the expression levels of these genes can be upregulated (activated) or downregulated (deactivated), thereby influencing the generation, maintenance, self-renewal, and/or differentiation of hematopoietic and/or stromal cell and/or stem cells. More specifically, CNTFR can affect survival, expansion or differentiation of neuronal cells or stem cells; TGF-β1, -β2 and -β3 affect cell survival, proliferation, differentiation, or induce apoptosis; Piwi-like-1 can affect cell division; C/EBP-α can affect lineage commitment; DMP-1 can affect differentiation to tooth cell-like cells; OASIS can affect osteoblast differentiation and/or maturation; Lhx-2 and HoxB4 can generate, expand or maintain hematopoietic stem cells; and Pax5 can affect lymphocyte development, neuronal cell development, or spermatogenesis.

Related to the regulation of the circadian clock system in accordance with the present invention is the ability to generate an in vitro engineered tissue that includes a plurality of cells or cell types in intimate contact with one another to form a tissue, with at least one of the cells or cell types having a circadian clock system that has been modulated to regulate growth and development of the at least one cell or cell type within the tissue. To the extent all cells or cell types in the engineered tissue have a circadian clock system, the circadian clock system of all cells or cell types can be modulated.

The tissue can be bone marrow, blood, blood vessel, lymph node, thyroid, parathyroid, skin, adipose, cartilage, tendon, ligament, bone, tooth, dentin, periodontal tissue, liver, nervous tissue, brain, spinal cord, retina, cornea, skeletal muscle, smooth muscle, cardiac muscle, gastrointestinal tissue, genitourinary tissue, bladder, pancreas, lung, or kidney tissues. The ex vivo development of bone marrow in a three-dimensional bioreactor of the type described above has been previously demonstrated (see, eg., U.S. patent application Ser. No. 09/715,852 to Wu et al., filed Nov. 17, 2000, and Ser. No.09/796,830 to Wu et al., filed Mar. 1, 2001, each of which is hereby incorporated by reference in its entirety).

The circadian clock system of cells in-vivo can be modulated using any of the various techniques described above, including without limitation: controlled light exposure, restricted feeding, administration of glucocorticoids or other molecules that can entrain or modulate the circadian clock. This includes factors produced by the SCN naturally, or molecules designed or discovered to act in a manner to modulate the circadian clock.

The circadian clock system for the cultured cells or cell types listed or engineered tissue can be modulated using any of the various techniques described above, including without limitation: co-culture with SCN cells, transfecting the one or more cell types of the culture or engineered tissue so they express one or more positive or negative regulators or a signaling molecule, introducing into the media one or more positive or negative regulators (as (TAT−) fusion proteins, RNA molecules, or signaling molecules for uptake (transduction) by the cell or cell types, or modifying the redox potential of the media (for example, by controlling oxygen levels, oxygen consumption rate with carbonyl cyanide m-chlorophenylhydrazone (CCCP) or adding lactate to the medium). Other methods for controlling the circadian gene expression include the feeding of media or serum in scheduled manner to entrain or modulate the circadian rhythm of cells in culture. This includes the use of gradients in concentration over time of entraining factors such as SCN conditioned media or media containing entraining factors such as SCN signaling molecules, glucocorticoids and other molecules that can entrain or modulate the circadian clock.

By virtue of controlling the circadian clock system of the tissue which is engineered in vitro, it is possible to produce a tissue that is appropriately developed and matured in cell type and number such that the cells are more readily adapted for introduction into a patient whose body has its own circadian clock system.

### EXAMPLES

The following examples are provided to illustrate embodiments of the present invention, but they are by no means intended to limit its scope.

The following materials and methods described below were employed in the research described in the Examples.

Housing of Animals

Male mice (Balb/c, 3-4 weeks old; Jackson Laboratory, Bar Harbor, Me.) were used to avoid interference by the female estral rhythm. Upon arrival, the mice were acclimated in the same room with a 12:12 light-dark cycle for at least two weeks prior to the initiation of the experiments. To diminish the disturbance of the sleep phase, the mice were housed 2 to 3 per cage. At each time point, bone marrow cells were harvested from the mice in one cage. The procedures were performed under a dim light during the dark phase of the light-dark cycle.

Bone Marrow Collection

Mice were sacrificed by cervical dislocation at Zeitgeber Time (ZT) 0, 4, 8, 12, 16 and 20. (At ZTO, the light was turned on and, at ZT12, the light was turned off.) In different studies, we initiated the experiments at either ZT 0 or 20 to eliminate differences caused by the sampling schedule. The femurs of individual mice were removed and the bone marrow cells were flushed with washing medium (McCoy's 5A; Gibco, Grand Island, N.Y.) supplemented with 1% fetal bovine serum (FBS; Hyclone, Logan, Utah). In certain experiments (Examples 1-2), 4-5 mice were sacrificed at each time point to ensure statistical significance. When RNA extraction was required, the bone marrow cells collected at each time point were lysed with the lysis buffer RLT (Qiagen, Valencia, Calif.) and stored at −70° C. prior to total RNA extraction (for less than one week) (Example 5).

Separation of Gr-1 Positive Cells:

Gr-1 positive cells were isolated by immunomagnetic bead separation using the CELLection Biotin Binder Kit (Dynal) following the manufacturer's protocol. Briefly, biotinylated rat anti-mouse Gr-1 monoclonal antibody (Pharmingen) was used to coat the streptavidin-conjugated magnetic polystyrene beads by incubating the mixture at room temperature for 30 minutes. 7×10⁶ bone marrow cells were mixed with 40 μl of the antibody coated beads and incubated at 4° C. for 30 minutes. The beads were then washed with washing medium and isolated using a magnet. Isolated cells were lysed directly on the beads for total RNA extraction. For each time point, 4-6 mice were sacrificed to ensure statistical significance.

Flow Cytometric Analysis of Gr-1 Positive Cells:

The purity of the immunomagnetically fractionated cell population was determined by flow cytometry in which the cell sample was incubated with a biotinylated rat anti-mouse Gr-1 monoclonal antibody (Pharmingen) at 4° C. for 30 minutes according to the manufacturer's instructions. The cells were washed with 1×phosphate-buffered saline (PBS; Gibco) and then incubated with an FITC-labeled goat anti-rat IgG polyclonal antibody (Pharmingen) at 4° C. for 30 minutes. The cells were then washed and resuspended in 1×PBS. For the negative control, the primary antibody was omitted. Percentages of Gr-1 positive cells were quantified by flow cytometry on an EPICS Profile Analyzer (Coulter) by analyzing 10,000 events.

Relative Quantitative Reverse Transcriptase-polymerase Chain Reaction (RT-PCR) (Examples 1 and 2):

For each RT-PCR experiment, samples from six time points were analyzed at the same time. Total RNA was purified from Gr-1 positive or unfractionated bone marrow cells using the RNeasy Mini Kit (Qiagen) according to the manufacturer's protocol. Following the DNase (Promega) treatment, approximately 2 μg of total RNA were reverse transcribed using Moloney murine leukemia virus reverse transcriptase (MMLV-RT; Stratagene) with random primers (Stratagene) at 37° C. for 60 minutes in a 20 μl reaction. The reverse transcriptase was then inactivated by incubation at 90° C. for 5 minutes. Internal control (Quantum RNA 18S Internal Standards; Ambion) was used according to the manufacturer's protocol to analyze the relative amount of mPer1 and mPer2 mRNA at different time points. The 18S non-productive competing primers (Competimer; Ambion) are designed to carry modified 3′ ends for blocking the extension by DNA polymerase. A 9:1 ratio of the 18S non-productive competing primers to the 18S primer mix was used to reduce the 18S cDNA signal to a level comparable to that of the target gene. The 18S cDNA and target cDNA (mPer1 or mPer2) were coamplified in a PCR-tube. Primers specific for mPer1 (PER1F, SEQ ID No: 10 and PER1R, SEQ ID No: 11) and mPer2 (PER2F, SEQ ID No: 12 and PER2R, SEQ ID No: 13) are shown in Table 1 below.

TABLE 1
Primers for RT-PCR of Per1 and Per2

| | | GenBank | Nucleotide |
|---|---|---|---|
| Primer | Sequence (5′→3′) | Accession | Position |
| PER1F | CCTCCACTGTATGGCCCAGACATGAGTG | AF022992 | 205 to 232 |
| PER1R | GCACTCAGGAGGCTGTAGGCAATGGAC | AF022992 | 524 to 550 |
| PER2F | CAGCAATGGCCAAGAGGAGTCTCACCGGAG | AF035830 | 1621 to 1650 |
| PER2R | CCGGGATGGGATGTTGGCTGGGAACTCGC | AF035830 | 1952 to 1980 |

Each GenBank Accession in Table 1 is hereby incorporated by reference in its entirety.

Within each PCR experiment, the linear range of amplification was first determined using cDNA pooled from 6 time points. PCR was performed with the Taq DNA polymerase (Advantage cDNA Polymerase Mix; Clontech) in 1×PCR reaction buffer (Clontech) containing 0.8 mM dNTPs under the following conditions: initial incubation at 94° C. for 3 minutes, 28-32 cycles (depending on the linear range) at 94° C. for 30 seconds, 60° C. for 45 seconds and 72° C. for 1 minute, followed by a 7 minutes extension at 72° C. As a negative control, the products of the RT reactions, without reverse transcriptase, were subjected to the same PCR amplification. The PCR products were resolved by electrophoresis on a 1.5% agarose gel (Gibco), stained with the fluorescent stain (GelStar; FMC), and their relative quantities were determined by using the Image-Pro Plus software (Media Cybernetics).

Differential Cell Counts:

Cytospin slides were prepared using a Cytospin centrifuge (Shandon, Sewickly, Pa.) by centrifuging 4×10⁴ cells/slide at 700 rpm for 5 min. Following centrifugation, slides were air-dried and stained with Wright's stain (Georetric Data, Wayne, Pa.) for 20 minutes followed by a distilled water wash for 2 minutes. Differential cell counts were performed blindly by counting over 100 cells per slide using a light microscope (Olympus, Melville, N.Y.).

Immunomagnetic Cell Sorting:

Bone marrow cells were incubated with ACK lysing buffer (0.15M NH₄Cl, 1 mM KHCO₃ and 0.1 mM Na₂EDTA; pH7.2) at room temperature for 4 minutes to remove red blood cells. The lin⁻ (lineage marker-negative) bone marrow cells were obtained by depleting lineage marker-positive cells using the MACS magnetic separation system (Miltenyi Biotec, Auburn, Calif.) according to the manufacturer's instructions. The antibodies used were PE-labeled rat anti-mouse Gr-1, TER119, B220, CD4, CD8, and Mac-1 monoclonal antibodies (all from BD PharMingen, San Diego, Calif.). Briefly, the cells were incubated with the antibody cocktail for the lineage markers described above at 6-10° C. for 15 minutes. After two washes with 1×phosphate-buffered saline (PBS; Sigma, St. Louis, Mo.) supplemented with 0.5% FBS (Hyclone), the cells were incubated with anti-PE antibody-coated magnetic beads (Miltenyi Biotec) at 6-10° C. for 15 minutes. The cells were then washed with 1×PBS (Sigma) supplemented with 0.5% FBS (Hyclone) and the positive cells were depleted using a magnetic column (Miltenyi Biotec).

Relative Quantitative Reverse Transcriptase-polymerase Chain Reaction (RT-PCR) (Example 3):

For each RT-PCR experiment, samples from six time points were analyzed at the same time. Total RNA was purified from the lin⁻ or unfractionated bone marrow cells using the RNeasy Mini Kit (Qiagen, Valencia, Calif.) according to the manufacturer's protocol. Total RNA purified from 10⁶ unfractionated bone marrow cells or 2×10⁵ lin⁻ cells was subjected to reverse transcription using SUPERSCRIPT II Reverse Transcriptase (Gibco) with random primers (Invitrogen, Carlsbad, Calif.) at 42° C. for 60 minutes in a 20-μl reaction. An internal control (Quantum RNA 18S Internal Standards; Ambion, Austin, Tex.) was used according to the manufacturer's protocol to analyze the relative amounts of mPer1, mClock, or GATA-2 mRNA at different time points. The 18S non-productive competing primers (Competimer; Ambion) are designed to carry modified 3′ ends for blocking extension by DNA polymerase. A 10:1 ratio of the 18S non-productive competing primers to the 18S primer mix was used to reduce the 18S cDNA signal to a level comparable to that of the target gene. The 18S cDNA and target cDNA (mPer1, mClcok, or GATA-2) were coamplified in the same PCR-tube.

Within each PCR experiment, the linear range of amplification was first determined using cDNA pooled from 6 time points. PCR was performed with Taq DNA polymerase (Advantage cDNA Polymerase Mix; Clontech, Palo Alto, Calif.) in 1×PCR reaction buffer (Clontech) containing 0.8 mM dNTPs under the following conditions (for mPer1, mClock, and the GATA-2 IG transcript): initial incubation at 94° C. for 3 minutes, 25-33 cycles (depending on the linear range) at 94° C. for 30 seconds, 60° C. for 30 seconds, and 72° C. for 30 seconds, followed by a 7-minute extension at 72° C. The PCR conditions for the GATA-2 IS transcript were initial incubation at 96° C. for 1 minute followed by 28-33 cycles (depending on the linear range) at 96° C. for 20 seconds and 68° C. for 1 minute. Primer sets used for RT-PCR were: forward and reverse for mPer1 (SEQ ID Nos: 10 and 14, respectively); forward and reverse primers for mPer2 (SEQ ID Nos: 15 and 16, respectively); forward and reverse primers for mClock (SEQ ID Nos: 17 and 18, respectively); forward and reverse primers for GATA-2 IG (SEQ ID Nos: 19 and 20, respectively); and forward and reverse primers for GATA-2 IS (SEQ ID Nos: 21 and 22, respectively) (as summarized in Table 2 below).

TABLE 2
Primers used for RT-PCR of mPer1, mPer2, mClock, GATA-2 IG, and
GATA-2 IS

| | | GenBank | Nucleotide |
|---|---|---|---|
| Target gene | Primer Sequence (5′→3′) | Accession | Position |
| mPer1 | CCTCCACTGTATGGCCCAGACATGAGTG | AF022992 | 205 to 232 |
| | ATGGGCTCTGTGAGTTTGTACTCTT | AF022992 | 496 to 520 |
| mPer2 | CAGCAATGGCCAAGAGGAGTC | AF035830 | 1621 to 1641 |
| | CCGGGATGGGATGTTGGCTGGGAACTC | AF035830 | 1950 to 1978 |
| mClock | ATGGTGTTTACCGTAAGCTGTAG | AF000998 | 389 to 411 |
| | CCAGTACTGTCGAATCTCACTAG | AF000998 | 666 to 688 |
| GATA-2 IG | CACCCCTATCCCGTGAATCCGCC | AF448814 | 1433 to 1455 |
| | AGCTGTGCTGGCTCCATGTAGTTAT | AB000096 | 246 to 270 |
| GATA-2 IS | TGGCCTAAGATCACCTCAACCATCG | AB009272 | 1638 to 1662 |
| | AGCTGTGCTGGCTCCATGTAGTTAT | AB000096 | 246 to 270 |

Each GenBank Accession in Table 2 is hereby incorporated by reference in its entirety.

As a negative control, the products of the RT reactions, without reverse transcriptase, were subjected to the same PCR amplification. The PCR products were resolved by electrophoresis on a 2% agarose gel (Gibco) and stained with a fluorescent stain (GelStar; FMC, Rockland, Me.). Their relative quantities were determined by using the Image-Pro Plus software (Media Cybernetics).

To test the effects of dexamethasone and phorbol-12-myristate-13-acetate (PMA) on mPer1 and mPer2 expression, aliquots of the lin⁻ cell were incubated with RPMI 1640 (Sigma) containing 200 nM dexamethasone (Sigma) or 1 μM PMA (Sigma) for 1 or 2 hours in a humidified 5% CO₂ incubator at 37° C. The control groups contained the same amounts of ethanol used to dissolve the respective reagent in the media. Relative quantitative RT-PCR was performed as described above.

Analysis of Mouse GATA-2 Gene 5′ Region:

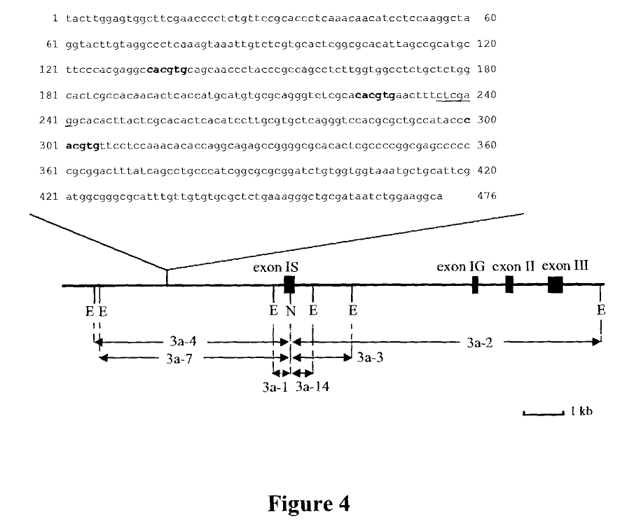

Phage DNA was purified from mouse genomic DNA clone 3a (a gift of Dr. Masayuka Yamamoto, Tohoku University, Japan), which contains the 5′ region of the mouse GATA-2 gene (Minegishi et al., “Alternative promoters regulate transcription of the mouse GATA-2 gene,” J. Biol. Chem. 273(6):3625-3634 (1998), which is hereby incorporated by reference in its entirety), and digested by Not I and partially digested by EcoR I for subcloning into the pBluescript II KS (−) vector (Stratagene, La Jolla, Calif.). Six distinct clones were obtained (FIG. 4). The isolated plasmids were then digested by restriction enzyme Pml I (New England Biolab, Beverly, Mass.) to identify and locate CACGTG (SEQ ID No: 2) E-boxes.

Transient Transfection Assay (Example 3 and 4):

Luciferase reporter constructs were generated as follows. The insert in clone 3a-7 was released by Kpn I and Sac I digestion and cloned into the same sites in pGL3-Basic (Promega, Madison, Wis.) to create pGL3-3a-7. The DNA fragment between the EcoR I site and the third Pml I site or the first Pml I site and the Xba I site (from 5′ to 3′) of pGL3-3a-7 was removed to generate pGL3-3a-31 or pGL3-3a-39, respectively. The pGL3-Elb reporter vector was derived from pG5E1b-Luc (Hsiao et al., “The linkage of Kennedy's neuron disease to ARA24, the first identified androgen receptor polyglutamine region-associated coactivator,” J. Biol. Chem., 274(29):20229-20234 (1999), which is hereby incorporated by reference in its entirety) by replacing the five GAL4 binding sites with the multiple cloning sites (from Kpn I to Xba I) of the pBluescript II KS (−) vector (Stratagene). The DNA fragment corresponding to nucleotides 76 to 351 in FIG. 4 was PCR-amplified and cloned into the EcoR I and BamH I sites of pGL3-E1b to generate pGL3-E1b-GEs. PCR by overlap extension was used to generate the same insert with individual or all E-box (CACGTG, SEQ ID No: 2) elements mutated to GGATTC (SEQ ID No: 23). The mutated inserts were then cloned into EcoR I-BamH I double digested pGL3-E1b to create pGL3-E1b-GEsM1, pGL3-E1b-GEsM2, pGL3-E1b-GEsM3, and pGL3-E1b-GEsM123. Nucleotides 76 to 223, 139 to 299, and 235 to 351 in FIG. 4 were amplified by PCR and cloned into the EcoR I and BamH I sites of pGL3-E1b to make pGL3-E1b-GE1, pGL3-E1b-GE2, and pGL3-E1b-GE3, respectively. Expression plasmids for mPER1, mPER2 and mPER3 (Jin et al., “A molecular mechanism regulating rhythmic output from the suprachiasmatic circadian clock,” Cell 96(1):57-68 (1999), which is hereby incorporated by reference in its entirety) were generously provided by Dr. Steven M. Reppert at Harvard Medical School. The hamster BMAL1 (hBMAL1) (Gekakis et al., “Role of the CLOCK protein in the mammalian circadian mechanism,” Science 280(5369):1564-1569 (1998), which is hereby incorporated by reference in its entirety) expression plasmid was kindly provided by Dr. Charles J. Weitz at Harvard Medical School. The full-length cDNA of mCLOCK (kindly provided by Dr. Joseph S. Takahashi, Northwestern University) was subcloned into pcDNA3 (Invitrogen). The mPER1ΔPAS expression plasmid was constructed by replacing the EcoR I-Cla I fragment of the mPER1 expression plasmid with the annealed oligos 5′-AATTCAGACATGAGTGGTCCCCTA-3′ (SEQ ID No: 24) and 5′-CGTAGGGGACCACTCATGTCTA-3′ (SEQ ID No: 25). The resulted expression construct excluded amino acids 6 to 515 of mPER1.

H1299 cells were maintained in RPMI1640 (Gibco) with 10% FBS (Hyclone). NIH3T3 cells were maintained in DMEM (Gibco) with 10% FBS (Hyclone). The day before transfection, 3×10⁵ cells/well were plated onto six-well plates. Cells were transfected with 500 ng of each expression plasmid, 100 ng of the firefly luciferase reporter construct and 2 ng of the Renilla luciferase control plasmid (pRL-SV40; Promega) using SuperFect transfection reagent (Qiagen) following the manufacturer's instructions. The Renilla luciferase control plasmid was cotrasfected to normalize transfection efficiency. When expression plasmids were omitted, same amount of the pcDNA3 plasmid was used to substitute the expression plasmids. Forty hours after transfection, cells were washed once with 1×PBS (Sigma) and lysed with 500 μl of passive lysis buffer (Promega). Luciferase activity of the cell lysate was assayed with the Dual-Luciferase Reporter Assay System (Promega) using a luminometer (Optocomp1; MGM Instruments) as recommended by the manufacturer.

RNA Arbitrarily Primed PCR (RAP-PCR) (Example 5):

Total RNA was purified from the bone marrow cells using the RNeasy Mini Kit (Qiagen) according to the manufacturer's instructions. RAP-PCR was performed using the RAP-PCR kit (Stratagene, La Jolla, Calif.) following the manufacturer's protocol. Following DNase (Promega, Madison, Wis.) treatment, 1 μg total RNA was used to synthesize first-strand cDNA with the random primer A2 (Stratagene) at 37° C. for 60 minutes. A quarter of the cDNA was then used for PCR with the same random primer at the following conditions: the first cycle at 94° C. for 1 minute, 36° C. for 5 minutes, and 72° C. for 5 minutes, followed by 40 cycles at 94° C. for 1 minute, 52° C. for 2 minutes, and 72° C. for 2 minutes. The PCR products were resolved on 7 M urea, 6% acrylamide gels and visualized by silver stain (Pharmacia, Piscataway, N.J.). Differentially displayed bands were excised, extracted from the gel, amplified, cloned, and sequenced. The DNA sequences were then compared to the various databases at GenBank using the BLASTn search program.

Relative Quantitative Reverse Transcriptase-polymerase Chain Reaction (RT-PCR) (Example 5):

For each RT-PCR experiment, samples from six time points were analyzed at the same time. Total RNA was purified from 2×10⁶ bone marrow cells using the RNeasy Mini Kit (Qiagen) according to the manufacturer's protocol. One sixth of the total RNA was reverse transcribed using Moloney murine leukemia virus reverse transcriptase (MMLV-RT; Stratagene) with random primers (Stratagene) at 37° C. for 60 minutes in a 20-μl reaction. An internal control (Quantum RNA 18S Internal Standards; Ambion, Austin, Texas) was used according to the manufacturer's protocol to analyze the relative amounts of the indicated mRNA at different time points. The 18S non-productive competing primers (Competimer; Ambion) are designed to carry modified 3′ ends for blocking the extension by DNA polymerase. A 9:1 ratio of the 18S non-productive competing primers to the 18S primer mix was used to reduce the 18S cDNA signal to a level comparable to that of the target gene. The 18S cDNA and target cDNA (6A-2-9, mlats2, or mlats2b) were coamplified in a PCR-tube. Primers used were Forward Primer 1 (SEQ ID No: 26) and Reverse Primer 4 (SEQ ID No: 31) for clone 6A-2-9, Forward Primer 1 (SEQ ID No: 26) and Reverse Primer 1 (SEQ ID No: 28) for mlats2, and Forward Primer 1 (SEQ ID No: 26) and Reverse Primer 2 (SEQ ID No: 29) for mlats2b, as shown in Table 3 below. Forward Primer 2 is SEQ ID No: 27 and Reverse Primer 3 is SEQ ID No: 30.

TABLE 3
PCR Primers for mlats, mlats2, mlats2b, and mlats2c

| | | GenBank | |
|---|---|---|---|
| Primer | Sequence (5′→3′) | Accession | Nucleotide Position |
| Forward | AAGGAAACTGGACTAACAATGAGGC | AB023958 | 116 to 140 in mlats2 |
| Primer 1 | | | |
| Forward | CACTGACACTGTTGACTGTTCTCT | AB023958 | 50 to 63 in mlats2 |
| Primer 2 | | | |
| Reverse | GGTCTGCTTGATGACTCGCACAATC | AB023958 | 574 to 598 in mlats2 |
| Primer 1 | | | |
| Reverse | GACACGCACCAGGAATATGCATCTG | AY015061 | 421 to 445 in mlats2b |
| Primer 2 | | | |
| Reverse | ACACGCACCAGGAATATGCATTGT | AY015062 | 568 to 591 in mlats2c |
| Primer 3 | | | |
| Reverse | ATCTGCCGGTTCACCTCTGCAGC | AB023958 | 416 to 438 in mlats2 |
| Primer 4 | | | |

Each GenBank Accession in Table 3 is hereby incorporated by reference in its entirety.

Within each PCR experiment, the linear range of amplification was first determined using cDNA pooled from 6 time points. PCR was performed with Taq DNA polymerase (Advantage cDNA Polymerase Mix; CLONTECH, Palo Alto, Calif.) in 1×PCR reaction buffer (CLONTECH) containing 0.8 mM dNTPs under the following conditions: initial incubation at 94° C. for 3 minutes, 25-30 cycles (depending on the linear range) at 94° C. for 30 seconds, 58° C. (for 6A-2-9 and mlats2) or 62° C. (for mlats2b) for 30 seconds and 72° C. for 30 seconds, followed by a 7-minute extension at 72° C. As a negative control, the products of RT reactions performed without reverse transcriptase were subjected to the same PCR amplification. The PCR products were resolved by electrophoresis on a 1.5% agarose gel (Gibco) and stained with fluorescent stain (GelStar; FMC, Rockland, Me.). Their relative quantities were then determined by using the Image-Pro Plus software (Media Cybernetics).

3′-Rapid Amplification of the cDNA End (RACE)

Total RNA was purified from the bone marrow cells using the RNeasy Mini Kit (Qiagen) according to the manufacturer's instructions. 3′-Rapid amplification of the cDNA end (RACE) was carried out using the SMART RACE cDNA Amplification Kit (CLONTECH) as suggested by the manufacturer. Briefly, the first-strand cDNA was synthesized using a primer containing a stretch of oligo(dT) and a universal primer binding sequence (CLONTECH). PCR was carried out using the Forward Primer 1 (Table 3 above) and the universal primer (CLONTECH) as follows: 5 cycles each at 94° C. for 5 seconds and 72° C. for 3 minutes; followed by 5 cycles each at 94° C. for 5 seconds, 70° C. for 10 seconds, and 72° C. for 3 minutes; and 30 cycles each at 94° C. for 5 seconds, 68° C. for 10 second and 72° C. for 3 minutes. The PCR products were cloned into the pCRII-TOPO TA cloning vector (Invitrogen, Carlsbad, Calif.) and their sequences determined using a model 373 AD DNA sequencer (Applied Biosystems).

Reverse Transcriptase-polymerase Chain Reaction (RT-PCR) (Example 6):

Following DNase (Promega) treatment, approximately 2 μg of total RNA from murine bone marrow cells was reverse transcribed using Moloney murine leukemia virus reverse transcriptase (MMLV-RT; Stratagene) with random primers (Stratagene) in a 20μl reaction. The resulting reaction mixture (2.5 μl) was used as a PCR template in a 25μl reaction using Taq DNA polymerase (AdvanTaq Plus DNA Polymerase; Clontech) under the following conditions: initial incubation at 94° C. for 3 minutes, 35 cycles each at 94° C. for 10 seconds, 58° C. for 30 seconds and 72° C. for 30 seconds, and the final incubation at 72° C. for 7 minutes. Primers used were Forward Primer 1 and Reverse Primer 1 for mlats2, Forward Primer 1 and Reverse Primer 2 for mlats2b and Forward Primer 2 and Reverse Primer 3 for mlats2c as shown in Table 3 above.

PCR Analysis of Gene Expression in Different Mouse Tissues (Example 5):

A PCR-based method was used to analyze the expression profiles of mlats2, mlats2b, and mlats2c in different mouse tissues using the RAPID-SCAN Gene Expression Panel (OriGene, Rockville, Md.). According to the manufacturer, the expression panel was prepared by isolating total RNA from different tissues of adult Swiss Webster mice. Poly-A⁺ RNA was then isolated and subjected to the first-strand cDNA synthesis using an oligo(dT) primer. Individual cDNA pools were confirmed to be free of genomic DNA contamination. For analysis of mlats2, mlats2b, and mlats2c expression, 1 ng of cDNA was used as the template for each tissue. The primer sets specific for individual splice variants are the same as described above. mlats2 and mlats2b were coamplified in the same PCR tube. The PCR conditions were the same as described above for RT-PCR. For β-actin, 1 pg of cDNA from each tissue and the β-actin primer set (OriGene) were used as suggested by the manufacturer.

Plasmid Construction:

pcDNA3-mLATS2 and pcDNA3-mLATS2N373 were generated by inserting the entire mLATS2 open reading frame (kindly provided by Dr. Hiroshi Nojima at Osaka University, Japan) or the BamH I-Not I fragment into the BamH I and Xho I sites or BamH I and Not I sites of pcDNA3 (Invitrogen), respectively. pGBKT7-mLATS2b was constructed by inserting the PCR-generated entire coding region of mlats2b into the Nde I and Sma I sites of pGBKT7 (CLONTECH) in frame with the GAL4 DNA binding domain. The same PCR product was also cloned into pcDNA3 to create pcDNA3-mLATS2b. pGBKT7-mLATS2 was generated by inserting the Bsm I-Xho I fragment of pcDNA3-mLATS2 into the Bsm I and Sal I sites of pGBKT7-mLATS2b. pGBKT7-mLATS2N373 was constructed by removing the Not I fragment from pGBKT7-mLATS2. pGBKT7-mLATS2N96 was constructed by removing the Pst I fragment from pGBKT7-mLATS2b. The coding region of mRBT1 was PCR-amplified using cDNA prepared from murine total bone marrow and cloned into the EcoR I and Pst I sites of pM (CLONTECH) in frame with the GAL4 DNA binding domain to generate pM-mRBT1. The primers used were 5′-TCGCCGGTTCATGGGAGGCTTAAAGAGG-3′ (SEQ ID No: 32) and 5′-GCGGCTGCAGCTTTAGGATCCCAGGAT-3′ (SEQ ID No: 33). The same PCR product was also cloned into the EcoR I and Sma I sites of pGADT7 (CLONTECH) in frame with the GAL4 activation domain to create pGADT7-mRBTI. pGADT7-mRBT1N121 was generated by removing the Xho I fragment from pGADT7-mRBT1. The PCR product encoding the C-terminal 76 amino acids of mRBT1 was cloned into the EcoR I and Sma I sites of pGADT7 to create pGADT7-mRBT1C76. The same PCR product was also cloned into the EcoR I and Pst I sites of pM to generate pM-mRBT1C76. pG5-E1b-LUC, in which 5 GAL4-binding sites and the E1b-minimal promoter are located upstream of the luciferase gene, was constructed as previously described (Hsiao et al., “The linkage of Kennedy's neuron disease to ARA24, the first identified androgen receptor polyglutamine region-associated coactivator,” J. Biol. Chem., 274(29):20229-20234 (1999), which is hereby incorporated by reference in its entirety).

Yeast Two-hybrid Assay:

Yeast two-hybrid screening was performed using the MATCHMAKER GAL4 Two-Hybrid System 3 (CLONTECH) and a human bone marrow MATCHMAKER cDNA library purchased from CLONTECH according to the manufacturer's instructions. Competent cells (AH109) were prepared as follows. YPD medium (2 ml; 2% peptone, 1% yeast extract, and 2% dextrose) was inoculated with a single colony and incubated overnight at 30° C. with shaking. The overnight culture (100 μl) was transferred into 25 ml of YPDA medium (YPD medium supplemented with 0.003% adenine) and grown overnight at 30° C. with shaking to the stationary phase. The overnight culture was then transferred into 150 ml of YPDA medium and grown for an additional 2 to 3 hours. The cells were harvested and washed once with 35 ml of sterile water. Finally, the cells were resuspended in 0.75 ml 1×TE/LiAc solution (10 mM Tris-HCl, 1 mM EDTA, and 0.1M lithium acetate, pH7.5). Cells were transformed with the bait and library plasmids as described in the manufacturer's manual. After transformation, cells were plated on quadruple dropout plates (-Ade/-His/-Leu/-Trp) to select for positive protein-protein interactions. Clones grown on the quadruple dropout plates were further confirmed by growth on plates containing X-alpha-Gal (CLONTECH) as blue colonies. The inserts of the positive clones were sequenced using a DNA sequencer (Perkin-Elmer ABI 377).

Mammalian One-hybrid Assay:

NIH3T3 cells were maintained in DMEM supplemented with 10% FBS (Hyclone). The day before transfection, 3×10⁵ cells/well were plated onto six-well plates. Cells were transfected with indicated amounts of the expression plasmid(s), 100 ng of pG5-E1b-LUC, and 4 ng of the Renilla luciferase control plasmid (pRL-SV40; Promega) using SuperFect transfection reagent (Quiagen). The Renilla luciferase control plasmid was cotransfected to normalize transfection efficiency. Plasmid pcDNA3 was added to bring the total amount of plasmid to 1.6 μg/well. Forty hours after transfection, cells were washed once with phosphate-buffered saline (PBS; Gibco) and lysed with 500 μl of passive lysis buffer (Promega). Luciferase activity was assayed with the Dual-Luciferase Reporter Assay System (Promega) using a luminometer (Optocomp1; MGM Instruments) as recommended by the manufacturer.

Southern Blot Analysis:

Mouse genomic DNA was purified from the bone marrow cells by the Genomic-tip 500 column (Qiagen) following the manufacturer's instructions. The genomic DNA (10 μg) was digested with Pst I and separated on a 0.8% agarose gel. The DNA was then transferred onto a positive-charged nylon membrane (Boehringer Mannheim) through capillary action. Southern blot analysis was performed using a digoxigenin-labeled probe generated by PCR (PCR DIG Probe Synthesis Kit; Boehringer Mannheim) following the manufacturer's protocol. Briefly, the membrane was blocked with blocking solution (Boehringer Mannheim) for 2 hours at 42° C. Hybridization was carried out at 42° C. overnight with DIG Easy Hyb hybridization buffer (Boehringer Mannheim) containing digoxigenin-labeled probes at a final concentration of 25 ng/ml. After hybridization, the membrane was washed twice, 5 minutes each, with 2× wash solution (2×SSC and 0.1% SDS) at room temperature, followed by additional two washes, 5 minutes each, with 0.5× wash solution (0.5×SSC and 0.1% SDS) at 68° C. Detection was performed using alkaline phosphatase-conjugated anti-digoxigenin antibodies and the chemiluminescent substrate CSDP (Boehringer Mannheim). Chemiluminescence was detected using an X-ray film (Kodak, Rochester, N.Y.).

### Example 1

Detection of Circadian Expression of mPer1 and mPer2 in Bone Marrow

First, a demonstration was made that both the mPer1 and mPer2 genes were expressed in bone marrow using RT-PCR with the primer sets (see Table 1 above) specific for mPer1 (PER1F, SEQ ID No: 10, and PER1R, SEQ ID No: 11) or mPer2 (PER2F, SEQ ID No: 12, and PER2R, SEQ ID No: 13). To examine the time-dependent and daily rhythmic expression of these two genes, an analysis was performed on their mRNA levels using relative quantitative RT-PCR with the same primer sets. To eliminate tube-to-tube variations, 18S rRNA was used as the internal control. Since the 18S rRNA is normally more abundant than the target mRNA, overamplification of the 18S rRNA is usually observed. To circumvent this problem, the 18S primers were mixed with the 18S non-productive competing primers (Competitor; Ambion), as described above, to reduce the PCR amplification efficiency of the 18S. Relative amounts of target mRNA at different time points were then compared after they were normalized to the 18S cDNA amplicons.

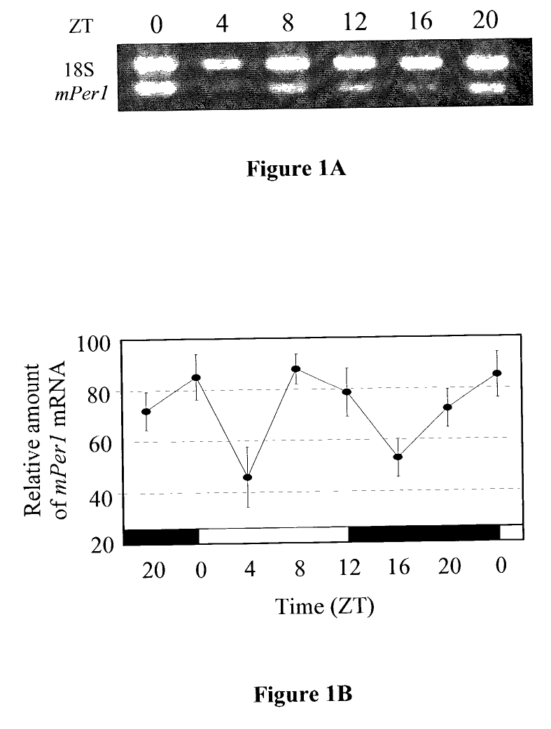

As expected, negative controls, which omitted reverse transcriptase in RT-PCR, did not yield any PCR products. Conversely, for the experimental runs, the RT-PCR product of mPer1 was detected in all the bone marrow samples taken at different time points. Furthermore, the amount of the mPer1 mRNA oscillated in a time-dependent manner (FIGS. 1A-B). The circadian variation reached statistical significance as determined by one way ANOVA (p<0.01). It exhibited two peaks at ZT 0 and ZT 8, respectively, over a 24-hour period. The peak-trough amplitude of the mPer1 RNA level was about 1.9-fold.

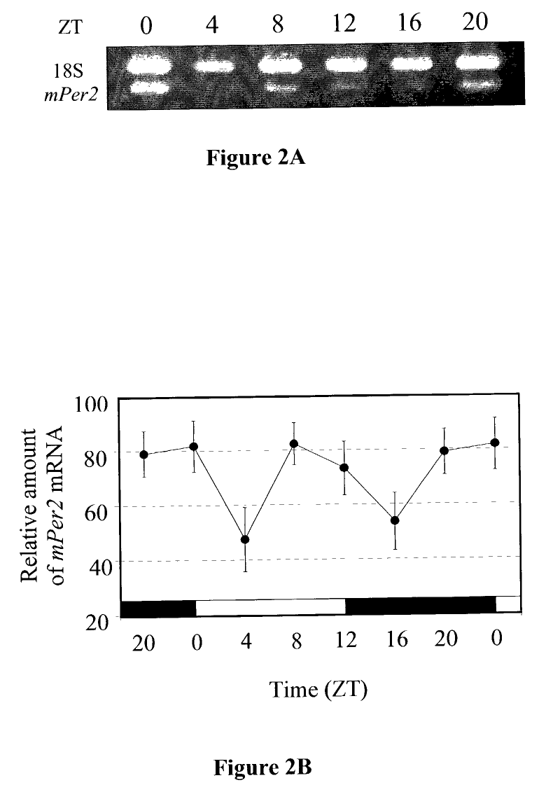

Similarly, the RT-PCR product of mPer2 was detected in all bone marrow samples and the levels of the mPer2 mRNA varied, over a 24-hour period (FIGS. 2A-B). The circadian variation showed a marginal statistical significance (one way ANOVA, p=0.07). Furthermore, it exhibited a similar pattern to that of the mPer1 expression with one peak between ZT 20-0 and another peak at ZT 8. The peak-trough amplitude of the mPer2 mRNA level was about 1.7-fold.

### Example 2

Circadian Expression of mPer1 and mPer2 in Gr-1 Positive Cells

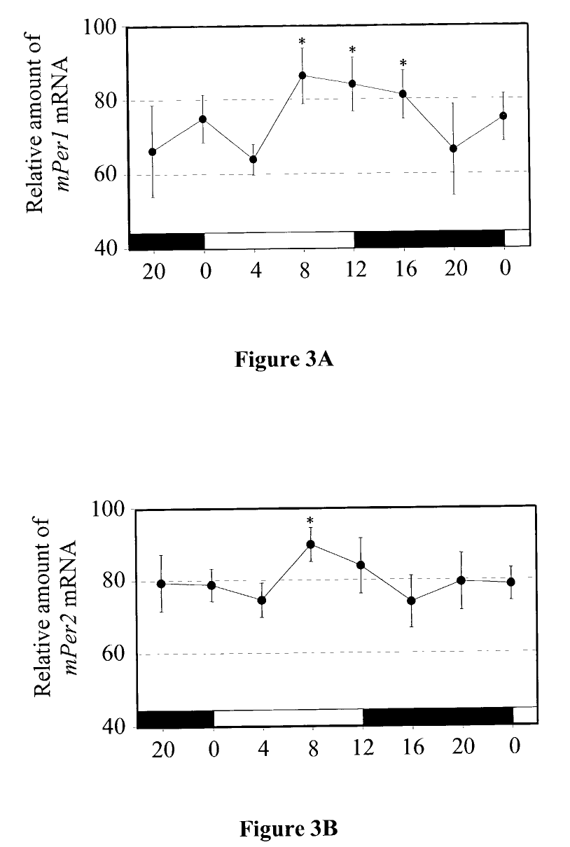

To investigate whether the expression patterns of the mPer1 and mPer2 are lineage-dependent, the mPer1 and mPer2 expression was examined in myeloid cells. Mycloid cells were purified using the anti-Gr-1 antibody-coated magnetic beads. Flow cytometry analysis demonstrated the purity of the Gr-1-positive fraction was close to 95%. Differential cell analysis based on cell morphology also confirmed that the Gr-1 positive fraction consisted of predominantly myeloid cells. The expression patterns of mPer1 (FIG. 3A) and mPer2 (FIG. 3B) in the Gr-1 positive fraction, in contrast to those in the unfractionated bone marrow cells, showed only a prominent peak at ZT 8 (t test, p<0.05). This result indicates that the circadian gene expression in bone marrow is lineage- and/or differentiation stage-specific.

Discussion of Examples 1 and 2

It has been reported that, in human, the serum concentrations of certain cytokines, including erythropoietin, tumor necrosis factor α, interleukin (IL)-2, IL-6, IL-10, and granulocyte-macrophage colony-stimulating factor (GM-CSF), vary over a 24-hour period (Sothem et al., “Circadian characteristics of interleukin-6 in blood and urine of clinically healthy men,” In Vivo 9:331-339 (1995); Young et al., “Circadian rhythmometry of serum interleukin-2, interleukin-10, tumor necrosis factor-alpha, and granulocyte-macrophage colony-stimulating factor in men,” Chronobiol. Int. 12:19-27 (1995); Wide et al., “Circadian rhythm of erythropoietin in human serum,” Br. J. Haematol. 72:85-90 (1989), each of which is hereby incorporated by reference in its entirety). However, there has been no direct evidence linking the circadian rhythms of hematopoiesis to the variations in cytokine concentrations in serum. In a recent study (Perpoint et al., “In vitro chronopharmacology of recombinant mouse IL-3, mouse GM-CSF, and human G-CSF on murine myeloid progenitor cells,” Exp. Hematol. 23:362-368 (1995), which is hereby incorporated by reference in its entirety), it was reported that the response of mouse CFU-GM to CSFs varied in a circadian pattern. Furthermore, the variations were independent of both the type and dose of the CSF tested. These findings indicate that the circadian rhythms of hematopoiesis are not merely a passive response to the variations of the cytokine concentrations in serum and that the marrow cells are subject to the control of an independent clock. However, whether an internal clock exists in bone marrow and whether the known clock components are expressed in the bone marrow cells remained unknown.

In Example 1 and 2, it was demonstrated that the murine bone marrow cells express mPer1 and mPer2, two known clock components. It was also shown that mPer1 expression oscillates robustly over a 24-hour period. Although the variation of mPer2 expression was less significant than that of mPer1 expression, the expression pattern of mPer2 was very similar to that of mPer1.

Unlike those in other tissues, the expression patterns of mPer1 and mPer2 in murine bone marrow exhibited two peaks in a 24-hour period. It has been shown that different cell lineages exhibit distinct circadian cycles as observed in the CFU assays and cell cycle analysis (Wood et al., “Distinct circadian time structures characterize myeloid and erythroid progenitor and multipotential cell clonogenicity as well as marrow precursor proliferation dynamics,” Exp. Hematol. 26:523-533 (1998), which is hereby incorporated by reference in its entirety). Consistently, the circadian expression patterns of mPer1 and mPer2 in Gr-1 positive cells are different from those for the unfractionated bone marrow. The Gr-1 positive cells mainly contribute to the second peak of the circadian gene expression, observed in the unfractionated bone marrow cells. It is plausible, therefore, to suggest that the circadian expression of mPer1 and mPer2 in the bone marrow is lineage- and/or differentiation stage-dependent.

It has been proposed that the output of the clock system is controlled via the clock-controlled genes (CCGs). In liver, DBP (albumin site D-binding protein), a transcription factor highly expressed in liver, has recently been shown to be a CCG (Ripperger et al., “CLOCK, an essential pacemaker component, controls expression of the circadian transcription factor DBP,” Genes Dev. 14:679-689 (2000), which is hereby incorporated by reference in its entirety). Its expression is under the control of the clock genes. In addition, several genes mediated by DBP are expressed in a cireadian manner (Lavery et al., “Circadian expression of the steroid 15 alpha-hydroxylase (Cyp2a4) and coumarin 7-hydroxylase (Cyp2a5) genes in mouse liver is regulated by the PAR leucine zipper transcription factor DBP,” Mol. Cell. Biol. 19:6488-6499 (1999), which is hereby incorporated by reference in its entirety). The clock system in liver therefore appears to mediate the circadian expression of the DBP gene, which in turn drives the circadian expression of the downstream target genes. The previously reported circadian variations in hematopoiesis and the oscillation of mPer1 and mPer2 in bone marrow, demonstrated in this work, indicate that a similar clock system very likely exists in bone marrow. It is therefore of great interest to identify CCGs in bone marrow and link the internal clock to the cellular activities of hematopoiesis.

The foregoing experimental work demonstrates, for the first time, the expression of the two known clock genes, mPer1 and mPer2, in murine bone marrow. Furthermore, they provide the evidence supporting the lineage- and/or differentiation stage-dependent circadian rhythms and the insights into the molecular mechanism that governs the circadian variations in hematopoiesis.

### Example 3

Circadian Expression of the Mouse GATA-2 Gene in Bone Marrow

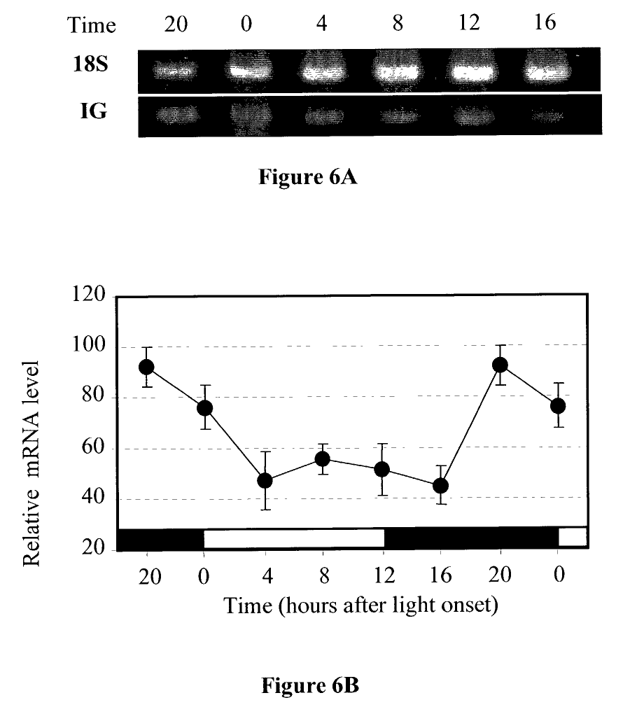

mGATA-2 has been shown to regulate proliferation and differentiation of hematopoietic stem/progenitor cells. Particularly, the expression level of mGATA-2 is critical for its function. Therefore, it was believed that mGATA-2 expression is modulated by the circadian clock in bone marrow. To test this hypothesis, the expression pattern of the mGATA-2 gene was examined over a 24-hour period in murine bone marrow. As reported previously (Minegishi et al., “Alternative promoters regulate transcription of the mouse GATA-2 gene,” J. Biol. Chem. 273(6):3625-3634 (1998), which is hereby incorporated by reference in its entirety), two distinct first exons (IS and IG) exist in the mGATA-2 gene. To distinguish the two transcripts containing distinct first exons (IS and IG transcripts), the primer set specific for the IS or IG transcript was used for the PCR analysis (see Table 2 above). In the total bone marrow, expression of the IG transcript oscillated significantly (p<0.05, one way ANOVA) and showed a circadian pattern, whereas the IS transcript was not detected (FIG. 6).

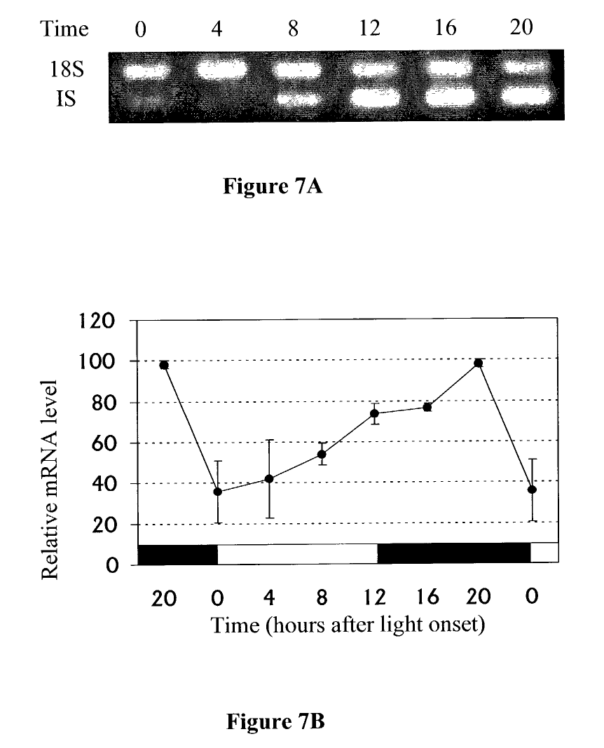

To determine the circadian expression profile of the IS transcript, lin⁻ cells were isolated from murine bone marrow by depleting lineage marker-positive cells as described above. Both the IS and IG transcripts were expressed in the lin⁻ cells obtained at different times of the light-dark cycle. Surprisingly, the expression level of the IG transcript did not oscillate within 24 hours. In contrast, expression of the IS transcript oscillated significantly (p<0.05, one way ANOVA) and showed a circadian pattern (FIG. 7). The mRNA level of the IS transcript peaked at 20 hours after light onset and the peak-trough amplitude was about 2.7-fold.

For comparison, the circadian expression profiles of mClock and mPer1 were also analyzed in the lin⁻ cells. mPer1 was expressed in a circadian manner with a prominent peak at 12 hours after light onset. On the other hand, there was no significant change in mClock expression in samples taken at different circadian times. The effects of dexamethasone and PMA on mPer1 and mPer2 expression in the lin⁻ cells was also examined. It was also demonstrated that dexamethasone and PMA can induce mPer1 expression and elicit circadian gene expression in cultured Rat-1 fibroblasts (Balsalobre et al., “Multiple signaling pathways elicit circadian gene expression in cultured Rat-1 fibroblasts,” Current Biology 10(20):1291-1294 (2000), which is hereby incorporated by reference in its entirety). In addition, dexamethasone can reset peripheral clocks in vivo through glucocorticoid receptors (Balsalobre et al., “Resetting of circadian time in peripheral tissues by glucocorticoid signaling,” Science 289(5488):2344-2347 (2000), which is hereby incorporated by reference in its entirety). While dexamethasone dramatically enhanced mPer1 expression in the lin⁻ cells, the expression level of mPer1 was not affected by PMA. On the other hand, neither dexamethasone nor PMA had a significant effect on mPer2 expression.

It is known that some clock-controlled genes are regulated directly by CLOCK and BMAL1 heterodimers through the CACGTG (SEQ ID No: 2) E-boxes in these genes (Jin et al., “A molecular mechanism regulating rhythmic output from the suprachiasmatic circadian clock,” Cell 96(1):57-68 (1999); Chen and Baler, “The rat arylalkylamine N-acetyltransferase E-box: differential use in a master vs. a slave oscillator,” Mol. Brain Res. 81(1-2):43-50 (2000); Ripperger et al., “CLOCK, an essential pacemaker component, controls expression of the circadian transcription factor DBP,” Genes Dev. 14:679-689 (2000), each of which is hereby incorporated by reference in its entirety). To determine whether the same mechanism could be responsible for circadian expression of the IS transcript in lin⁻ cells, the 5′ region of the mouse GATA-2 gene was analyzed using restriction enzyme Pml I, which specifically recognizes the CACGTG (SEQ ID No: 2) motif. Three E-boxes were identified at about 3 kbp upstream of exon IS (FIG. 4). No other E-boxes were found in the region analyzed in the current study.

### Example 4

Positive and Negative Regulation of mGATA-2 Gene Expression

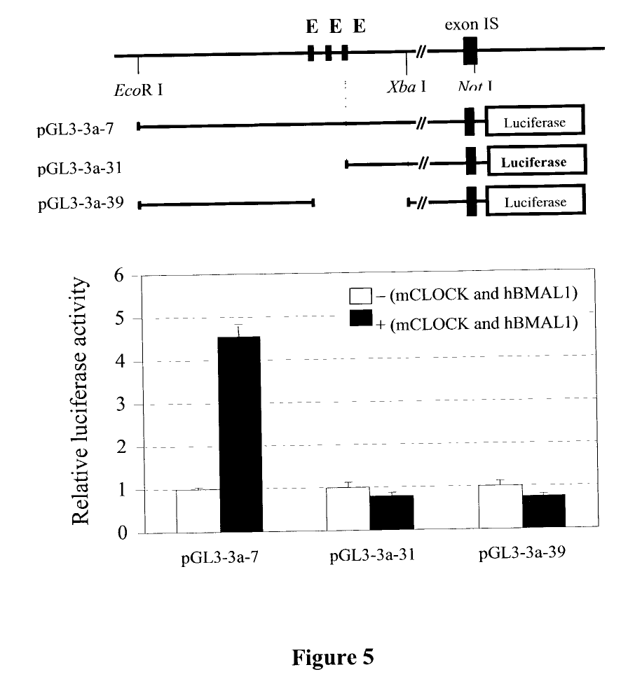

To directly examine the ability of CLOCK and BMAL1 heterodimers to activate mGATA-2 gene expression, a 4.5-kbp DNA fragment corresponding to part of exon IS and its promoter region were cloned into a promoterless luciferase reporter vector (pGL3-Basic) (FIG. 5). Two deletion mutants were also constructed for comparison. In the presence of mCLOCK and hBMAL1, expression of the wild-type reporter construct was increased by 4.5-fold (FIG. 5). In contrast, CLOCK and BMAL1-induced transcriptional activation was completely abolished upon removal of the three E-boxes and the flanking regions (FIG. 5).

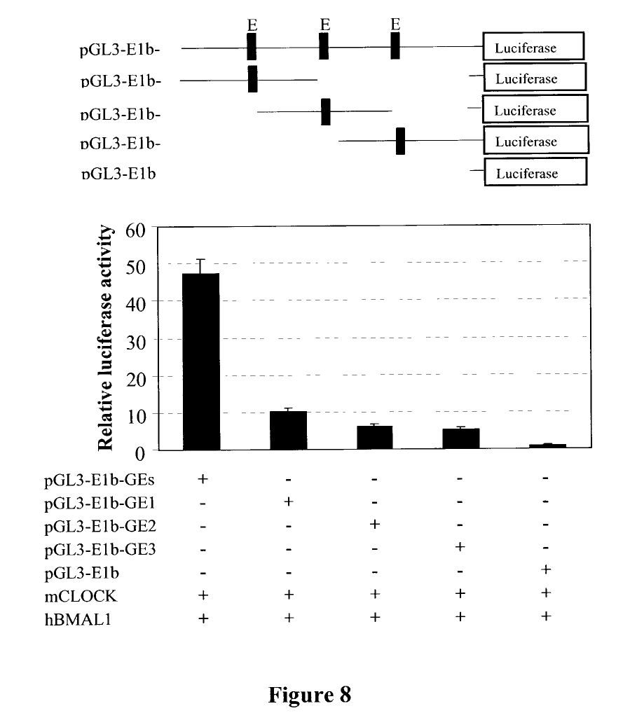

To further study the function of the three E-boxes, a 275-bp DNA fragment harboring the three E-boxes was cloned, as well as the individual E-boxes and their flanking regions (70-80 bp each sites), into the E1b minimal promoter-containing vector (pGL3-E1b; FIG. 8). In the presence of CLOCK and BMAL1, each E-box construct had a substantial increase over the control, in which no E-box was present (5-to 10-fold induction; FIG. 8). In addition, the three E-boxes together elicited a 47.5-fold increase in CLOCK and BMAL1-mediated transcription (FIG. 8).

Both mCLOCK and hBMAL1 were required for the induction. Consistently, mutation of the individual E-boxes reduced transcriptional activation by CLOCK and BMAL1 heterodimers (27.5% to 56.8% of the value from the wild type construct). Mutation of all three E-boxes completely blocked the enhancer activity of the 275-bp DNA fragment (compared to the control reporter vector pGL3-E1b). Taken together, these results show that CLOCK and BMAL1 acted through the three E-boxes to activate gene expression.

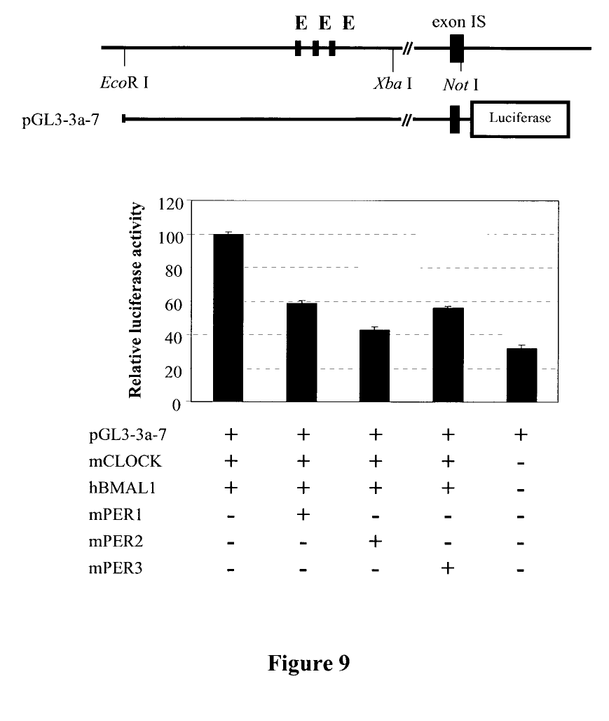

It has been shown that the negative regulators (e.g., PER1, PER2, and PER3) of the circadian clock inhibit CLOCK and BMAL1-mediated expression of clock-controlled genes in the transient transfection assay (Jin et al., “A molecular mechanism regulating rhythmic output from the suprachiasmatic circadian clock,” Cell 96(1):57-68 (1999); Kume et al., “mCRY1 and mCRY2 are essential components of the negative limb of the circadian clock feedback loop,” Cell 98(2):193-205 (1999), each of which is hereby incorporated by reference in its entirety). To further confirm the CLOCK and BMAL1-dependent activation of the mGATA-2 gene, cells were cotransfected with mPER1, mPER2, or mPER3 (the negative regulators of the circadian clock) expression plasmid. As shown in FIG. 9, mPER1, mPER2, and mPER3 each significantly inhibited CLOCK and BMAL1-mediated transcription of the reporter gene through the IS promoter. Similarly, CLOCK and BMAL1-dependent transcriptional activation through the three E-boxes was also inhibited by the PER proteins. The inhibitory effect of PER proteins was specific as deletion of the PAS domain abolished the inhibitory effect of mPER1.

Discussion of Examples 3 and 4

Circadian variations in different aspects of hematopoiesis have been documented (Laerum, “Hematopoiesis occurs in rhythms,” Exp. Hematol. 23:1145-1147 (1995); Smaaland, “Circadian rhythm of cell division,” Prog. Cell. Cycle. Res. 2:241-266 (1996), each of which is hereby incorporated by reference in its entirety). However, the molecular mechanisms governing the rhythms are still unknown. As shown in Examples 1 and 2, the circadian expression profiles of mPer1 and mPer2 in murine bone marrow indicate the presence of a clock system in bone marrow to locally regulate hematopoiesis. To further extend these studies, an analysis of mPer1 and mClock expression in the lin⁻ bone marrow cells was performed. The data are consistent with the characteristics of the circadian clock in that: 1) the mPer1 mRNA level oscillates within 24 hours; 2) the mClock mRNA level does not change significantly (Okano et al., “Cloning of mouse BMAL2 and its daily expression profile in the suprachiasmatic nucleus: a remarkable acceleration of Bmal2 sequence divergence after Bmal gene duplication.” Neurosci. Lett. 300(2):111-114 (2001); Yagita et al., “Molecular mechanisms of the biological clock in cultured fibroblasts,” Science 292(5515):278-281 (2001), each of which is hereby incorporated by reference in its entirety); and 3) expression of mPer1 is regulated by the glucocorticoid signaling pathway (Balsalobre et al., “Multiple signaling pathways elicit circadian gene expression in cultured Rat-1 fibroblasts,” Current Biology 10(20):1291-1294 (2000); Balsalobre et al., “Resetting of circadian time in peripheral tissues by glucocorticoid signaling,” Science 289(5488):2344-2347 (2000), each of which is hereby incorporated by reference in its entirety). Thus, a functional clock system appears to exist in lin⁻ bone marrow cells.

mGATA-2 was examined to determine whether it is a clock-controlled gene in bone marrow. The circadian expression patterns of both IS and IG transcripts in murine bone marrow were determined using relative quantitative RT-PCR. The IS transcript was shown to be expressed in a circadian manner in the lin⁻ bone marrow cells. In contrast, the expression level of the IG transcript did not oscillate at different times. It has been shown that expression of the IS and IG transcripts are controlled by two distinct promoters (Minegishi et al., “Alternative promoters regulate transcription of the mouse GATA-2 gene,” J. Biol. Chem. 273(6):3625-3634 (1998), which is hereby incorporated by reference in its entirety). While the IG transcript is expressed in bone marrow and several non-hematopoietic tissues, such as heart, kidney, and ovary, the IS transcript is only detected in bone marrow (Minegishi et al., “Alternative promoters regulate transcription of the mouse GATA-2 gene,” J. Biol. Chem. 273(6):3625-3634 (1998), which is hereby incorporated by reference in its entirety). Since both the IS and IG transcripts encode the same protein, it is possible that expression of mGATA-2 is subject to circadian control only in primitive hematopoietic cells.

While the IG transcript was detected in both total bone marrow and lin⁻ cells, the IS transcript was only found in the lin⁻ cells. These data are in agreement with a human study, in which the human IS transcript was only detected in CD34⁺ bone marrow cells, although the human IG transcript was observed in both total and CD34⁺ bone marrow cells (Pan et al., “Identification of human GATA-2 gene distal IS exon and its expression in hematopoietic stem cell fractions,” J. Biochem. 127(1):105-112 (2000), which is hereby incorporated by reference in its entirety). Since both transcripts are not expressed in the lineage marker-positive cells (Minegishi et al., “Alternative promoters regulate transcription of the mouse GATA-2 gene,” J. Biol. Chem. 273(6):3625-3634 (1998), which is hereby incorporated by reference in its entirety), it appears that expression of the IS transcript is restricted to even more primitive hematopoietic cells. Despite the fact that the IG transcript did not oscillate in lin⁻ bone marrow cells, its expression level was rhythmic in a circadian manner in total bone marrow cells. One explanation for these findings is that the number of IG transcript-expressing cells varies in murine bone marrow over the course of 24 hours.

In addition to the circadian expression pattern of the mGATA-2 IS transcript in lin⁻ bone marrow cells, three functional E-boxes in the IS promoter were identified in the context of the transient transfection assay. CLOCK and BMAL1enhanced transcription through the wild-type IS promoter, but not the truncated promoters lacking the three E-boxes. Furthermore, it was demonstrated that each E-box mediated CLOCK and BMAL1-dependent transcriptional activation. These findings indicate that the mGATA-2 gene is a direct target of CLOCK and BMAL1 heterodimers in bone marrow.

Several lines of evidence strongly suggest that the balance/combination of various hematopoietic transcription factors, rather than the presence or absence of a master regulator, controls lineage commitment in hematopoiesis (Sieweke and Graf, “A transcription factor party during blood cell differentiation,” Curr. Opin. Genetics & Development 8(5): 545-551 (1998); Orkin, “Hematopoietic stem cells: molecular diversification and developmental interrelationships,” in Stem Cell Biology, Marshak et al., Eds., Cold Spring Harbor Laboratory Press (2001), p. 289, each of which is hereby incorporated by reference in its entirety). Several lineage-facilitated transcription factors are co-expressed in the multipotential progenitors prior to commitment to individual lineages (Cheng et al., “Temporal mapping of gene expression levels during the differentiation of individual primary hematopoietic cells,” Proc. Nat'l Acad. Sci. USA 93(23):13158-13163 (1996); Tsang et al., “FOG, a multitype zinc finger protein, acts as a cofactor for transcription factor GATA-1 in erythroid and megakaryocytic differentiation,” Cell 90(1):109-119 (1997); Andrews et al., “Erythroid transcription factor NF-E2 is a haematopoietic-specific basic-leucine zipper protein,” Nature 362(6422):722-728 (1993); Scott et al., “Requirement of transcription factor PU.1 in the development of multiple hematopoietic lineages,” Science 265(5178):1573-1577 (1994); Sposi et al., “Cell cycle-dependent initiation and lineage-dependent abrogation of GATA-1 expression in pure differentiating hematopoietic progenitors,” Proc. Natl. Acad. Sci. USA 89(14):6353-6357 (1992), each of which is hereby incorporated by reference in its entirety). Consistently, multilineage gene expression has been shown to precede lineage commitment (Hu et al., “Multilineage gene expression precedes commitment in the hemopoietic system,” Genes & Development 11(6):774-785 (1997), which is hereby incorporated by reference in its entirety). Some hematopoietic transcription factors, such as GATA-1, PU.1, and C/EBP, exert their actions in combination with others (Tsang et al., “FOG, a multitype zinc finger protein, acts as a cofactor for transcription factor GATA-1 in erythroid and megakaryocytic differentiation,” Cell 90(1):109-119 (1997); Nerlov and Graf, “PU.1 induces myeloid lineage commitment in multipotent hematopoietic progenitors,” Genes Dev. 12(15):2403-2412 (1998); Nerlov et al., “Distinct C/EBP functions are required for eosinophil lineage commitment and maturation,” Genes Dev. 12(15):2413-2423 (1998), each of which is hereby incorporated by reference in its entirety). In some cases, hematopoietic transcription factors form large protein complexes (Wadman et al., “The LIM-only protein Lmo2 is a bridging molecule assembling an erythroid, DNA-binding complex which includes the TAL1, E47, GATA-1 and Ldb1/NLI proteins,” EMBO J. 16(11):3145-3157 (1997), which is hereby incorporated by reference in its entirety) and individual transcription factors may engage in different protein complexes along the differentiation process to turn on different genes (Sieweke and Graf, “A transcription factor party during blood cell differentiation,” Curr. Opin. Genetics & Development 8(5): 545-551 (1998), which is hereby incorporated by reference in its entirety). In addition, negative cross-regulation between lineage-affiliated transcription factors has been demonstrated. For example, PU.1 and GATA-1 negatively regulate each other through direct protein-protein interaction (Zhang et al., “Negative cross-talk between hematopoietic regulators: GATA proteins repress PU.1,” Proc. Natl. Acad. Sci. USA 96(15):8705-8710 (1999); Zhang et al., “PU.1 inhibits GATA-1 function and erythroid differentiation by blocking GATA-1 DNA binding,” Blood 96(8):2641-2648 (2000); Nerlov et al., “GATA-1 interacts with the myeloid PU.1 transcription factor and represses PU.1-dependent transcription,” Blood 95(8):2543-2551 (2000); Rekhtman et al., “Direct interaction of hematopoietic transcription factors PU.1 and GATA-1: functional antagonism in erythroid cells,” Genes Dev. 13(11):1398-1411 (1999), each of which is hereby incorporated by reference in its entirety). Therefore, a subtle change in the amounts of specific transcription factors can exhibit important effects on critical protein-protein interactions. Indeed, concentration-dependent effects of hematopoictic transcription factors, such GATA-1, PU.1, and GATA-2, have been documented (Heyworth, et al., “A GATA-2/estrogen receptor chimera functions as a ligand-dependent negative regulator of self-renewal,” Genes Dev. 13(14): 1847-60 (1999); McDevitt et al., “A ‘knockdown’ mutation created by cis-element gene targeting reveals the dependence of erythroid cell maturation on the level of transcription factor GATA-1,” Proc. Natl. Acad. Sci. USA 94(13):6781-6785 (1997); DeKoter and Singh, “Regulation of B lymphocyte and macrophage development by graded expression of PU.1,” Science 288(5470):1439-1441 (2000), each of which is hereby incorporated by reference in its entirety). Therefore, up-regulation and/or down-regulation of some lineage-affiliated transcription factors may disturb the balance and result in lineage commitment. The above data support the idea that oscillation of hematopoietic transcription factors can be controlled by the clock components. They therefore suggest that hematopoiesis is modulated by the circadian clock.

In summary, the above data indicate that mGATA-2 is a clock-controlled gene in bone marrow. As a transcription factor expressed in hematopoietic stem and progenitor cells, mGATA-2 is believed to drive circadian expression of its target genes and thus adapt the resulting hematopoietic activities to the day-night cycle.

### Example 5

Identification and Characterization of mlats2, a Potential Clock-Controlled Gene in Murine Bone Marrow

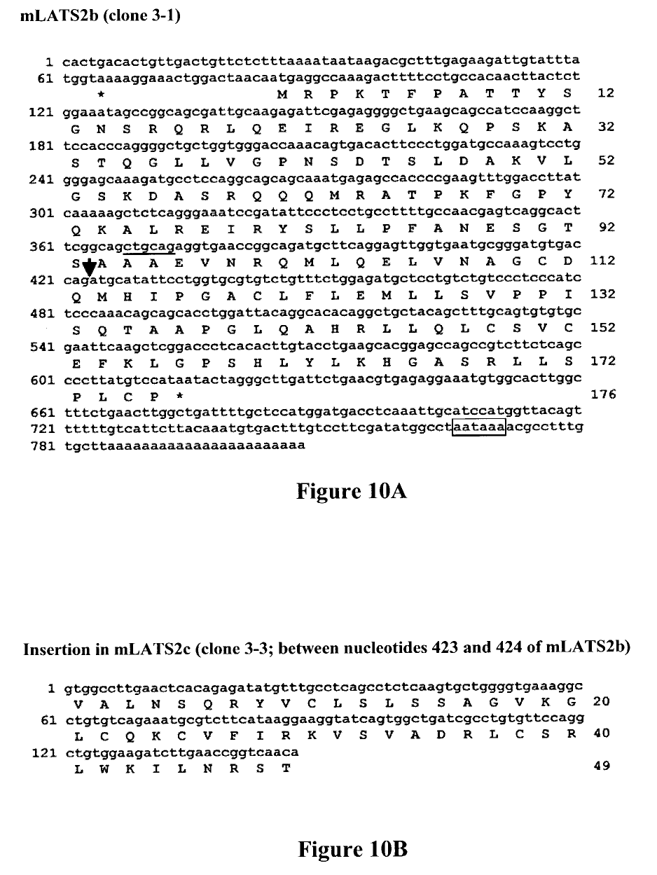

Total murine bone marrow cells were collected at 6 different circadian times for direct comparison of gene expression patterns using the RNA arbitrarily primed PCR technique. DNA bands that showed circadian oscillation were excised from the gel for determination of their sequences. A cDNA (6A-2-9) encoding a polypeptide homologous to cell cycle regulator hLATS1 was cloned. The circadian expression pattern of 6A-2-9 was confirmed by relative quantitative RT-PCR. The open reading frame of 6A-2-9 contains a putative start codon, but the 3′ end was not complete. The attempt to clone full-length cDNA of this gene using the 3′-RACE technique employing a primer corresponding to the putative start codon (Forward Primer 1, SEQ ID No: 26, Table 3 above) revealed two distinct cDNA fragments. Two PCR products of about 750 and 890 base pairs, respectively, were obtained. Subsequently, it was found that the cDNA clone 6A-2-9 indeed codes for part of mLATS2 (Yabuta et al., “Structure, expression, and chromosome mapping of LATS2, a mammalian homologue of the Drosophila tumor suppressor gene lats/warts,” Genomics 63(2):263-270 (2000), which is hereby incorporated by reference in its entirety). However, the 3′-RACE products are much shorter than the reported mlats2 cDNA (>3000 bp). The first 357 base pairs (nucleotides 67-423, FIG. 10A) of the originally cloned 3′-RACE products, namely clones 3-1 and 3-3, are identical to the 5′ region of mlats2 (nucleotides 116 to 472, GenBank Accession AB023958, which is hereby incorporated by reference in its entirety). The 5′ identical region (nucleotides 1-66 in FIG. 10A) of clones 3-1/3-3 was obtained by PCR employing Forward Primer 2 (SEQ ID No: 27) paired with Reverse Primer 2 (SEQ ID No: 29, clone 3-1) or Reverse Primer 3 (SEQ ID No: 30, clone 3-3) (see Table 3 above). The poly-adenylation signal AATAAA (SEQ ID No: 34) is found 14 bp upstream from the poly-A tail of clones 3-1 and 3-3 (FIG. 10A). When compared to mLATS2 (GenBank Accession BAA92380, which is hereby incorporated by reference in its entirety), the deduced amino acid sequences of clones 3-1 and 3-3 contain the same N-terminal 113 residues as those of mLATS2 but distinct C-termini (FIG. 10C). Furthermore, clone 3-3 contains an in-frame insertion of 49 amino acids not found in mLATS2 or clone 3-1.

Sequence alignment among mlats2, hlats2/kpm, clones 3-1/3-3, and the corresponding human genomic DNA sequence (GenBank Accession NT_{—}009917, which is hereby incorporated by reference in its entirety) shows a putative intron located at between nucleotides 716 and 717 of hlats2/kpm. The putative splice site corresponds to nucleotides 423 and 424 of clones 3-1/3-3, representing the exact location where the identity between mlats2 and clones 3-1/3-3 breaks off (FIG. 10A). The putative splice donor and acceptor in the human genomic DNA sequence conform to the GT/AG rule (Stephens and Schneider, “Features of spliceosome evolution and function inferred from an analysis of the information at human splice sites,” J. Mol. Biol. 228(4):1124-1136 (1992), each of which is hereby incorporated by reference in its entirety). Since the nucleotide sequences of mlats2 and hlats2/kpm are well conserved in this region, it is most likely that nucleotides 472 and 473 of mlats2 (GenBank Accession AB023958; corresponding to nucleotides 423 and 424 of clones 3-1/3-3, respectively) are also at the exon-intron boundaries. In addition, the fact that the 5′ regions, including a portion of the 5′ untranslated region (5′ UTR), in all three transcripts are identical further supports that clones 3-1 and 3-3 are derived from alternative splicing of the mlats2 gene. To further ascertain whether mlats2 is a single copy gene in the mouse genome, Southern blot analysis was carried out using a probe within the region common to mlats2, clone 3-1 and clone 3-3 (nucleotides 67 to 389 in clone 3-1). Based on the comparison between human genomic DNA and the mlats2 cDNA, it appears that the sequence covered by the probe is located in one exon. Therefore, a single band would be expected on the Southern blot if mlats2, clone 3-1, and clone 3-3 are derived from the same gene. Upon performing the Southern hybridization, a single band of about 1.6 kb was observed.

In addition, the mlats2 gene has been located in the central region of mouse chromosome 14 by interspecific mouse backcross mapping (Yabuta et al., “Structure, expression, and chromosome mapping of LATS2, a mammalian homologue of the Drosophila tumor suppressor gene lats/warts,” Genomics 63(2):263-270 (2000), which is hereby incorporated by reference in its entirety). Taken together, it appears that clones 3-1 and 3-3 are the alternatively spliced forms of mlats2. These two novel splice variants are hereafter named mlats2b and mlats2c, respectively.

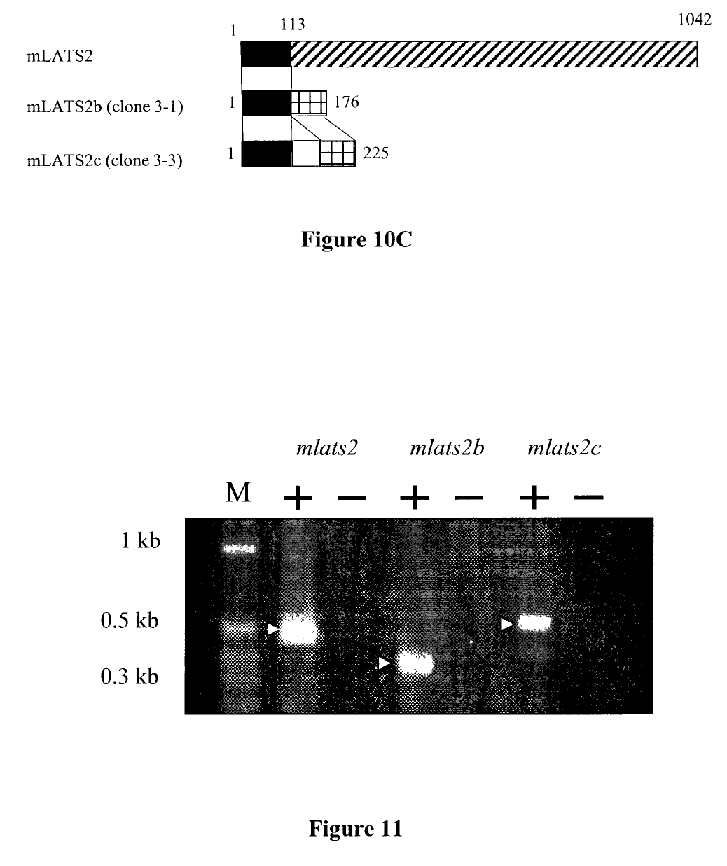

Expression of mlats2, mlats2b, and mlats2c in murine bone marrow was confirmed by RT-PCR employing primer sets specific for individual transcripts. PCR products of expected sizes (483 bp for mlasts2, 379 bp for mlats2b, and 525 bp for mlats2c) were obtained (FIG. 11). All PCR products were sequenced to confirm their identities. The same PCR primer pairs were used to examine the expression of mlats2, mlats2b, and mlats2c in various mouse tissues. mlats2 was expressed in most tissues analyzed with the highest level observed in testis. Conversely, expression in thymus was very low. Similarly, mlats2b was also widely expressed. However, the ratios of the expression level of mlats2 to that of mlats2b appear to be tissue-specific. In particular, in brain, spleen and testis, expression of mlats2 was much higher than that of mlats2b. In contrast, in thymus and lung, the reversed pattern was observed. Expression of mlats2c was relatively weak in all tissues except liver, in which the expression level of mlats2c was comparable to those of mlats2 and mlats2b.

### Example 6

Circadian Expression Profiles of mLats2 and mLats2b

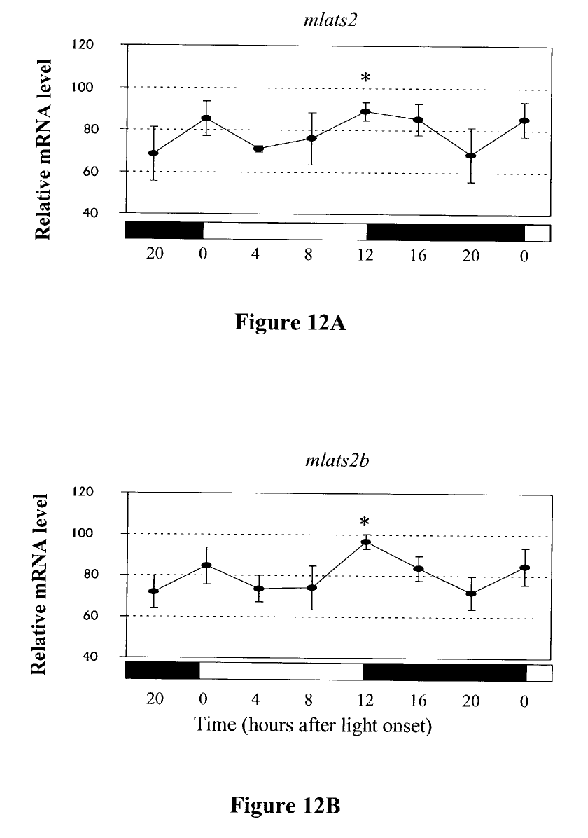

Although the initial relative quantitative RT-PCR result confirmed the circadian expression pattern of clone 6A-2-9 obtained from the RAP-PCR screening, the primer set used for the analysis amplified all three transcripts, mlats2, mlats2b, and mlats2c. To determine the circadian expression profiles of mlats2 and mlats2b individually, relative quantitative RT-PCR was performed using primer sets specific for mlats2 or mlats2b, respectively. As shown in FIGS. 12A-B, the circadian expression profiles of mlats2 and mlats2b were very similar. Both oscillated over the course of 24 hours and peaked at 12 hours after light onset. When the circadian expression patterns of mlats2 and mlats2b were compared to that of clone 6A-2-9, both similarity and discrepancy were observed. The mean values at 0 and 12 hours after light onset were always higher than those at their preceding and subsequent time points. However, the expression level of clone 6A-2-9 exhibited a peak at time 0. Therefore, it is possible that one or more splice variants remain to be identified. Alternatively, mlats2c could be highly expressed at time 0.

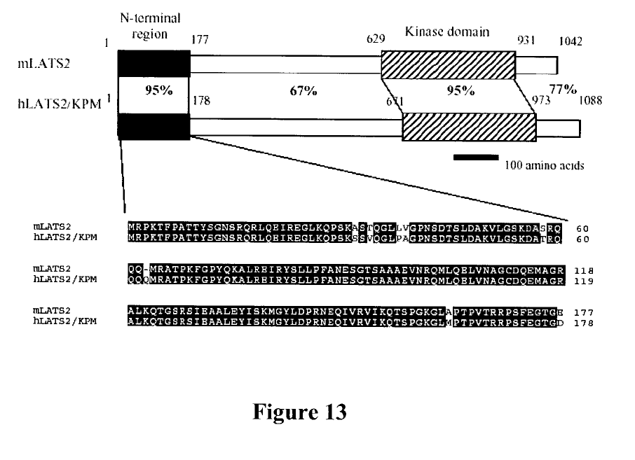

The kinase domain located near the C-terminus of LATS2 is highly conserved between human and mouse proteins. It is noteworthy that the other highly conserved region is the N-terminal domain of LATS2 (FIG. 13). It is possible that this region is important for protein-protein interaction. It is therefore interesting that mLATS2b has the same N-terminus as that of mLATS2, while lacking the kinase domain. It is plausible that the role of mLATS2b is to modulate the function of mLATS2 via competitive binding to a target protein. To elucidate the role of mLATS2b, I searched for its potential-interaction partners using yeast two-hybrid screening. A total of 47 positive clones were obtained after screening more than 10⁶ clones of the human bone marrow cDNA library using mLATS2b as a bait. The genes and number of clones identified (in parenthesis) are as follows: RBT1 (1); RACK1 (8); ABP-280 (7); eIF3 subunit 5 (2); DRAL/SLIM3/FHL2 (2); proapoptosis caspase adaptor protein (1); thymidine kinase (1); tenascin XA (1); lysosomal proteinase cathepsin B (1); succinate dehydrogenase (1); glutamine synthase (1); vanyl-tRNA synthetase 2 (1); fibulin 5 (1); sorcin (1); ribosomal protein L17 (1); mitofilin (1); lysyl oxidase (1); arylsulfatase A (1); peroxiredoxin 2 (1); and 13 others encoding unidentified proteins.

These potential mLATS2b-interacting proteins include proteins involved in translation, cytoskeleton remodeling, signal transduction, and metabolic pathways. One of these proteins, the Replication Protein Binding Trans-Activator (RBT 1), previously identified as a transcriptional co-activator associated with Replication Protein A (Cho et al., “RBT1, a novel transcriptional co-activator, binds the second subunit of replication protein A,” Nucl. Acids Res. 28(18):3478-3485 (2000), which is hereby incorporated by reference in its entirety), is particularly interesting because it may play a role in the regulation of DNA replication.

The interaction between mRBT1 and mLATS2/2b was further characterized by the yeast two-hybrid assay. As expected, mLATS2 also interacted with mRBT1. Since a comparable result was obtained with only the N-terminal 373 amino acids of mLATS2 (mLATS2N373), the kinase domain is not needed for the interaction between mRBT1 and mLATS2. The N-terminal 96 amino acids of mLATS2/2b (mLATS2N96), however, did not interact with mRBT1. The N-terminal 121 amino acids of mRBT1 (mRBT1N121) could interact with mLATS2, mLATS2N373, and mLATS2b but not with mLATS2N96. In contrast, the C-terminal 76 amino acids of mRBT1 (mRBT1C76), which contains the transactivation domain, did not interact with mLATS2/2b. Considering the fact that mLATS2 and mLATS2b share the same N-terminal 113 amino acids, the data shown here suggest that the RBT1-interacting region of mLATS2/2b is located in the common region and the peptide corresponding to amino acids 96 and 113 is essential for the interaction.

As RBT1 has a transactivation domain located in its C-terminal region (Cho et al., “RBT1, a novel transcriptional co-activator, binds the second subunit of replication protein A,” Nucl. Acids Res. 28(18):3478-3485 (2000), which is hereby incorporated by reference in its entirety), the effects of mLATS2 and mLATS2b on RBT1 were determined in the context of the mammalian one-hybrid assay. Consistent with the previous report (Cho et al., “RBT1, a novel transcriptional co-activator, binds the second subunit of replication protein A,” Nucl. Acids Res. 28(18):3478-3485 (2000), which is hereby incorporated by reference in its entirety), when fused to the GAL4 DNA binding domain, both full-length and C-terminal 76 amino acids of mRBT1 showed high levels of transcriptional activity (>1000 fold when compared with GAL4 alone) in the context of the mammalian one-hybrid assay (data not shown). In the presence of mLATS2, the transcriptional activity of mRBT1 was significantly inhibited. The inhibitory effect of mLATS2 was exerted on RBT1because the transcriptional activity of the GAL4 DNA-binding domain was not affected by mLATS2. Furthermore, the inhibitory effect of mLATS2 on mRBT1 was dependent on their interaction since the activity of the mRBT1 C-terminal 76 amino acids (mRBT1C76), which did not interact with mLATS2 in the yeast two-hybrid assay, was not negatively regulated by mLATS2. Deletion of the kinase domain completely abolished the inhibitory effect of mLATS2 on the transcriptional activity of mRBT1. Finally, the inhibitory effect of mLATS2 on mRBT1 transcriptional activity was antagonized by mLATS2b.

Discussion of Examples 5 and 6

The clock-controlled genes in murine bone marrow were demonstrated by a comparison of gene expression patterns at six circadian times. A cDNA fragment corresponding to the 5′ region of mlats2 was cloned based on its circadian expression. lats2 as well as lats1 (Yabuta et al., “Structure, expression, and chromosome mapping of LATS2, a mammalian homologue of the Drosophila tumor suppressor gene lats/warts,” Genomics 63(2):263-270 (2000); Tao et al., “Human homologue of the Drosophila melanogaster lats tumour suppressor modulates CDC2 activity,” Nature Genetics 21(2):177-181 (1999); Nishiyama et al., “A human homolog of Drosophila warts tumor suppressor, h-warts, localized to mitotic apparatus and specifically phosphorylated during mitosis,” FEBS Letters 459(2):159-165 (1999); Hori et al., “Molecular cloning of a novel human protein kinase, kpm, that is homologous to warts/lats, a Drosophila tumor suppressor,” Oncogene 19:3101-3109 (2000), each of which is hereby incorporated by reference in its entirety) are mammalian homologues of the warts/lats gene that was first identified as a tumor suppressor gene in Drosophila (Xu et al., “Identifying tumor suppressors in genetic mosaics: the Drosophila lats gene encodes a putative protein kinase,” Development 121(4):1053-1063 (1995), which is hereby incorporated by reference in its entirety). Several lines of evidence indicate the involvement of LATS1 and LATS2 in cell cycle regulation. For example, it has been shown that phosphorylation of hLATS1 is cell cycle-dependent and the phosphorylated hLATS1 negatively regulates CDC2 activity by forming the hLATS1-CDC2 complex in the mitotic phase (Tao et al., “Human homologue of the Drosophila melanogaster lats tumour suppressor modulates CDC2 activity,” Nature Genetics 21(2):177-181 (1999), which is hereby incorporated by reference in its entirety). High incidence of soft-tissue sarcomas and ovarian stromal cell tumors in the lats1^{-/-} mice also supports the role of LATS1 in cell cycle control (St. John et al., “Mice deficient of Lats1 develop soft-tissue sarcomas, ovarian tumours and pituitary dysfunction,” Nature Genetics 21(2): 182-186 (1999), which is hereby incorporated by reference in its entirety). In addition, when introduced into lats1-deficient cells, hLATS1 causes cell cycle arrest in the G2/M phase through the inhibition of CDC2 kinase activity (Yang et al., “Human homologue of Drosophila lats, LATS1, negatively regulate growth by inducing G(2)/M arrest or apoptosis,” Oncogene 20(45):6516-6523 (2001), which is hereby incorporated by reference in its entirety). Similarly, the human KPM protein (identical to hLATS2) has been shown to undergo phosphorylation during the mitotic phase and has been suggested to play a role in the progression of mitosis (Hori et al., “Molecular cloning of a novel human protein kinase, kpm, that is homologous to warts/lats, a Drosophila tumor suppressor,” Oncogene 19:3101-3109 (2000), which is hereby incorporated by reference in its entirety). Furthermore, expression of hLATS2 is induced by p53, a tumor suppressor gene involved in cell cycle control (Kostic and Shaw, “Isolation and characterization of sixteen novel p53 response genes,” Oncogene 19(35):3978-3987 (2000), which is hereby incorporated by reference in its entirety). Therefore, it is believed that the bone marrow clock can regulate cell proliferation through mLATS2, which in turn causes the circadian variations in the cell cycle status of bone marrow cells.

Two splice variants, mlats2b and mlats2c, encoding shorter versions of mLATS2, were identified. One important function of alternative splicing is to produce a functional variant by including or excluding domains important for protein-protein interaction, transcriptional activation or catalytic activity. In particular, several cell cycle regulators are expressed in different forms as a result of alternative splicing. For example, three splice variants of the human CDC25B have been identified and shown to exhibit different phosphatase activities in vivo (Baldin et al., “Alternative splicing of the human CDC25B tyrosine phosphatase. Possible implications for growth control?” Oncogene 14(20):2485-2495 (1997), which is hereby incorporated by reference in its entirety). Another example is p10, an alternatively spliced form of the human p15 cyclin-dependent kinase (CDK) inhibitor. In contrast to p15, p10 does not bind to CDK4 or CDK6 (Tsuburi et al., “Cloning and characterization of p10, an alternatively spliced form of p15 cyclin-dependent kinase inhibitor,” Cancer Res. 57(14):2966-2973 (1997), which is hereby incorporated by reference in its entirety). In addition, the respective splice variants of cyclin C, D1, and E, which have distinct expression patterns and functions, have been reported (Li et al., “Alternatively spliced cyclin C mRNA is widely expressed, cell cycle regulated, and encodes a truncated cyclin box,” Oncogene 13(4):705-712 (1996); Sawa et al., “Alternatively spliced forms of cyclin D1 modulate entry into the cell cycle in an inverse manner,” Oncogene 16(13):1701-1712 (1998); Sewing et al., “Alternative splicing of human cyclin E,” J. Cell Science 107(Pt 2):581-588 (1994); Mumberg et al., “Cyclin ET, a new splice variant of human cyclin E with a unique expression pattern during cell cycle progression and differentiation,” Nucl. Acids Res. 25(11):2098-2105 (1997), each of which is hereby incorporated by reference in its entirety). Comparison between mLATS2, mLATS2b, and mLATS2c (FIG. 10C) revealed that they have the same N-terminal 113 amino acids. However, the kinase domain is missing in mLATS2b and mLATS2c, which strongly suggests that mLATS2b and mLATS2c could regulate the function of mLATS2 by competitively binding to the same target protein. This possibility was addressed by the identification of proteins that interact with mLATS2/2b. The yeast two-hybrid assays revealed that mRBT1 can interact with both mLATS2 and mLATS2b. In addition, mLATS2 inhibited the transcriptional activity of mRBT1 in the context of the mammalian one-hybrid assay, and the inhibitory effect of mLATS2 was antagonized by mLATS2b. Collectively, these data demonstrate that mLATS2b is a negative regulator of mLATS2.

The fact that mLATS2 can negatively regulate mRBT1 further supports a role of mLATS2 as a cell cycle regulator. As a replication protein A (RPA)-interacting protein, it is possible that RBT1 promotes cell proliferation. Indeed, the expression levels of hRBT1 are higher in cancerous cells in comparison to non-transformed cells (Cho et al., “RBT1, a novel transcriptional co-activator, binds the second subunit of replication protein A,” Nucl. Acids Res. 28(18):3478-3485 (2000), which is hereby incorporated by reference in its entirety). In addition, transactivation of RBT1 is significantly down-regulated by p53 (Cho et al., “RBT1, a novel transcriptional co-activator, binds the second subunit of replication protein A,” Nucl. Acids Res. 28(18):3478-3485 (2000), which is hereby incorporated by reference in its entirety), although it remains to be determined whether p53 acts through LATS2 to inhibit RBT1.

In summary, mlats2 was identified as a clock-controlled gene in murine bone marrow. In addition, it was demonstrated that mLATS2 is negatively regulated by mLATS2b, a mLATS2 isoform generated by alternative splicing. Based on the above evidence and the well documented circadian variations in the cell cycle status of bone marrow cells, it is believed that mLATS2 as a cell cycle regulator.

### Example 7

Regulation of Per1 Promoter-Induced Transcription Using Neurotransmitters

A Per1-luciferase reporter plasmid was constructed essentially as described above, using a 7.2 kb fragment of the promoter region from mper1, forming pGL3-mPer1-7.2 kb. NIH 3T3 cells were transfected with pGL3-mPer1-7.2 kb as described above and cells were exposed to 10⁻⁶ M forskolin as a positive control, 10⁻⁶ M isoproterenol (a beta-adrenergic agonist), 10⁻⁶ M propranolol (a beta-adrenergic antogonist), 10⁻⁶ M phenylephrine (an alpha-adrenergic agonist), and 10⁻⁶ M pentolamine (an alpha-adrenergic antagonist). Cells were exposed to the neurotransmitters for 7 hours and luciferase activity was measured as described above.

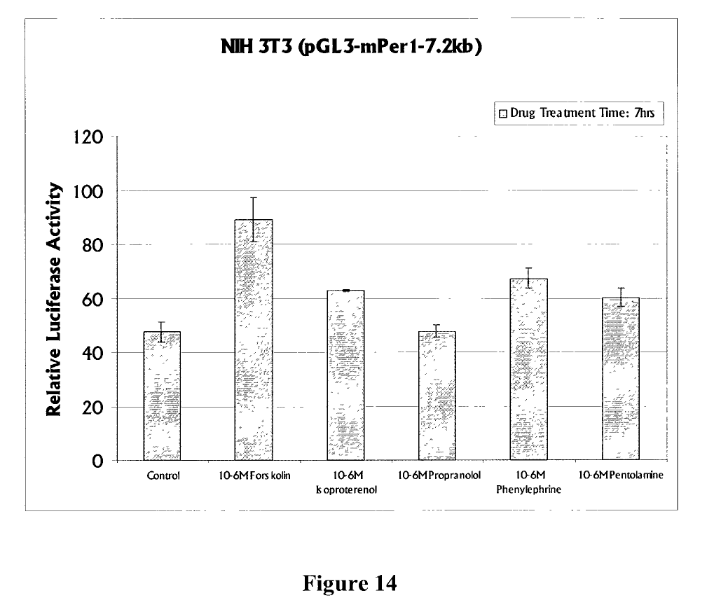

As shown in FIG. 14, each of the neurotransmitters analogs isoproterenol, phenylephrine, and 1 pentolamine showed increased luciferase activity relative to control (although expression levels were slightly diminished relative to the forskolin positive control). These results demonstrate that several different neurotransmitters likely act on the mper1 promoter region to induce transcriptional activity.

Recent evidence suggests that peripheral clocks are entrained by humoral signals regulated by the SCN. For example, circadian expression of Per2 in peripheral tissues is abolished in SCN-lesioned rats (Sakamoto et al., “Multitissue circadian expression of rat period homolog (rPer2) mRNA is governed by the mammalian circadian clock, the suprachiasmatic nucleus in the brain,” J. Biol. Chem. 273:27039-27042 (1998), which is hereby incorporated by reference in its entirety). In addition, a serum shock causes an immediate induction of Per1 and Per2 followed by circadian expression of these two genes as well as other clock-dependent genes including Dbp, Tef, and Rev-Erbα in cultured Rat-1 fibroblasts (Balsalobre et al., “A serum shock induces circadian gene expression in mammalian tissue culture cells,” Cell 93:929-937 (1998), which is hereby incorporated by reference in its entirety). Several factors, including forskolin (an activator of adenylate cyclase), phorbol-12-myristate-13-acetate (PMA; an activator of protein kinase C), and dexamethasone, induce immediate Per1 up-regulation and trigger circadian expression of Per1, Per2, Cry1, Dbp, and Rev-Erbα in cultured Rat-1 fibroblasts (Balsalobre et al., “Multiple signaling pathways elicit circadian gene expression in cultured Rat-1 fibroblasts,” Current Biology 10(20):1291-1294 (2000); Balsalobre et al., “Resetting of circadian time in peripheral tissues by glucocorticoid signaling,” Science 289(5488):2344-2347 (2000), each of which is hereby incorporated by reference in its entirety). Furthermore, injection of dexamethasone into mice resets the circadian clocks in various peripheral tissues without affecting the central clock in the SCN (Balsalobre et al., “Resetting of circadian time in peripheral tissues by glucocorticoid signaling,” Science 289(5488):2344-2347 (2000), which is hereby incorporated by reference in its entirety). Taken together, these data indicate that expression of Per1 in peripheral tissues is regulated by multiple signaling pathways and, as observed in the SCN, induction of Per1 is the initial event associated with clock resetting.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of controlling bone marrow cell development, said method comprising:
providing bone marrow cells having a circadian clock system and
manipulating the circadian clock system under conditions effective to control bone marrow cell development.

2. The method according to claim 1, wherein the method is carried out in vitro.

3. The method according to claim 1, wherein the method is carried out in vivo.

4. The method according to claim 1, wherein the bone marrow cells are stem cells.

5. The method according to claim 4, wherein the stem cells are selected from the group consisting of totipotent stem cells, pluripotent stem cells, myeloid stem cells, mesenchymal stem cells, and lymphoid stem cells.

6. The method according to claim 1, wherein the bone marrow cells are bone marrow progenitor cells.

7. The method according to claim 6, wherein the bone marrow progenitor cells are selected from the group consisting of CFU-GEMM cells, Pre B cells, lymphoid progenitors, prothymocytes, BFU-E cells, CFU-Meg cells, CFU-GM cells, CFU-G cells, CFU-M cells, CFU-E cells, and CFU-Eo cells.

8. The method according to claim 1, wherein the bone marrow cells are bone marrow precursor cells.

9. The method according to claim 8, wherein the bone marrow precursor cells are selected from the group consisting of promonocytes, megakaryoblasts, myeloblasts, monoblasts, normoblast, myeloblasts, proerythroblasts, B-lymphocyte precursors, and T-lymphocyte precursors.

10. The method according to claim 1 wherein bone marrow cells are selected from the group consisting of natural killer cells, dendritic cells, bone cells, tooth cells, B-lymphocytes, T-lymphocytes, and macrophages.

11. The method according to claim 1, wherein the bone marrow cells develop into cells selected from the group consisting of blood cells, liver cells, neural cells, muscle cells, chondrocytes, cartilage cells, bone cells, tooth cells, fat cells, hematopoietic support cells, pancreatic cells, cornea cells, retinal cells, and heart muscle cells.

12. The method according to claim 1, wherein bone marrow cells are manipulated to activate bone marrow cell development.

13. The method according to claim 1, wherein the bone marrow cells are manipulated to deactivate bone marrow cell development.

14. The method according to claim 1, wherein said manipulating comprises exposing the bone marrow cells to a medium comprising suprachiasmatic nucleus cells.

15. The method according to claim 14, wherein the suprachiasmatic nucleus cells are SCN2.2 cells.

16. The method according to claim 1, wherein said manipulating comprises exposing the bone marrow cells to a medium comprising one or more circadian signal molecules or one or more positive or negative regulators.

17. The method according to claim 16, wherein the one or more circadian signal molecules are selected from the group consisting of glucocorticoids, neurotransmitters, SCN cell signaling molecules, redox potential modulators, and combinations thereof.

18. The method according to claim 16, wherein the bone marrow cells are present in a medium comprising a positive regulator, a negative regulator, or a combination thereof.

19. A method of controlling stem cell self-renewal, differentiation and/or functions, said method comprising:
providing stem cells having a circadian clock system and
manipulating the circadian clock system under conditions effective to control stem cell self-renewal, differentiation and/or functions.

20. The method according to claim 19, wherein the method is carried out in vitro.

21. The method according to claim 19, wherein the method is carried out in vivo.

22. The method according to claim 19, wherein the stem cells are selected from the group consisting of totipotent stem cells, pluripotent stem cells, myeloid stem cells, mesenchymal stem cells, neural stem cells, liver stem cells, muscle stem cells, fat tissue stem cells, skin stem cells, limbal stem cells, hematopietic stem cells, AGM (aorta-gonad-mesonephros) stem cells, yolk sac stem cells, bone marrow stem cells, embryonic stem cells, embryonic germ cells, and lymphoid stem cells.

23. The method according to claim 19, wherein stem cell self-renewal is activated.

24. The method according to claim 19, wherein stem cell self-renewal is deactivated.

25. The method according to claim 19, wherein stem cell differentiation is activated.

26. The method according to claim 19, wherein stem cell differentiation is deactivated.

27. The method according to claim 19, wherein the stem cells develop into cells selected from the group consisting of blood cells, liver cells, neural cells, muscle cells, chondrocytes, cartilage cells, bone cells, tooth cells, fat cells, hematopoietic support cells, pancreatic cells, cornea cells, retinal cells, and heart muscle cells.

28. The method according to claim 19, wherein said manipulating comprises exposing the stem cells to a medium comprising suprachiasmatic nucleus cells.

29. The method according to claim 28, wherein the suprachiasmatic nucleus cells are SCN2.2 cells.

30. The method according to claim 19, wherein said manipulating comprises exposing the stem cells to a medium comprising one or more circadian signal molecules or one or more positive or negative regulators.

31. The method according to claim 30, wherein the one or more circadian signal molecules are selected from the group consisting of glucocorticoids, neurotransmitters, SCN cell signaling molecules, redox potential modulators, and combinations thereof.

32. The method according to claim 30, wherein the stem cells are present in a medium comprising a positive regulator, a negative regulator, or a combination thereof.

33. An in vitro engineered tissue comprising:
a plurality of cells or cell types in intimate contact with one another to form a tissue, the cells or cell types having a circadian clock system that has been modulated to regulate growth, development, and/or functions of the cells or cell types within the tissue.

34. The engineered tissue according to claim 33, wherein the tissue is bone marrow, blood, blood vessel, lymph node, thyroid, parathyroid, skin, adipose, cartilage, tendon, ligament, bone, tooth, dentin, periodontal tissue, liver, nervous tissue, brain, spinal cord, retina, cornea, skeletal muscle, smooth muscle, cardiac muscle, gastrointestinal tissue, genitourinary tissue, bladder, pancreas, lung or kidney.

35. The engineered tissue according to claim 33, wherein the plurality of cells or cell types are present in a medium comprising suprachiasmatic nucleus cells.

36. The engineered tissue according to claim 35, wherein the suprachiasmatic nucleus cells are SCN2.2 cells.

37. The engineered tissue according to claim 34, wherein the plurality of cells or cell types are present in a medium comprising one or more circadian signal molecules or one or more positive or negative regulators.

38. The engineered tissue according to claim 37, wherein the one or more circadian signal molecules are selected from the group consisting of glucocorticoids, neurotransmitters, SCN cell signaling molecules, redox potential modulators, and combinations thereof.

39. The engineered tissue according to claim 37, wherein the plurality of cells or cell types are present in a medium comprising a positive regulator, a negative regulator, or a combination thereof.

40. A method of controlling expression of a clock controlled gene, said method comprising:
providing a cell having a circadian clock system and
manipulating the circadian clock system of the cell under conditions effective to alter expression of a clock controlled gene selected from the group consisting of GATA-2, IL-12, IL-16, GM-CSF, LATS2, BMP-2, BMP-4, TERT, TGF-β1, TGF-β2, TGF-β3, Piwi-like-1, CEBP-α, DMP-1, OASIS, Lhx2, HoxB4, Pax5, and CNTFR.

41. The method according to claim 40, wherein the method is carried out in vitro.

42. The method according to claim 40, wherein the method is carried out in vivo.

43. The method according to claim 40, wherein said manipulating comprises exposing the cell to medium comprising suprachiasmatic nucleus cells.

44. The method according to claim 43, wherein the suprachiasmatic nucleus cells are SCN2.2 cells.

45. The method according to claim 40, wherein said manipulating comprises exposing the cell to a medium comprising one or more circadian signal molecules or one or more positive or negative regulators.

46. The method according to claim 45, wherein the one or more circadian signal molecules are selected from the group consisting of glucocorticoids, neurotransmitters, SCN cell signaling molecules, redox potential modulators, and combinations thereof.

47. The method according to claim 45, wherein the plurality of cells or cell types are present in a media comprising a positive regulator, a negative regulator, or a combination thereof.

48. The method according to claim 40, wherein the cell is a stem cell.

49. The method according to claim 48 wherein the stem cell is selected from the group consisting of totipotent stem cells, pluripotent stem cells, myeloid stem cells, mesenchymal stem cells, neural stem cells, liver stem cells, muscle stem cells, fat tissue stem cells, skin stem cells, limbal stem cells, hematopietic stem cells, AGM (aorta-gonad-mesonephros) stem cells, yolk sac stem cells, bone marrow stem cells, embryonic stem cells, embryonic germ cells, and lymphoid stem cells.

50. The method according to claim 48, wherein the clock controlled gene is GATA-2.

51. The method according to claim 50, wherein said manipulating activates GATA-2 expression.

52. The method according to claim 50, wherein said manipulating deactivates GATA-2 expression.

53. The method according to claim 50, wherein said manipulating alters GATA-2 expression to influence stem cell self-renewal or differentiation.

54. The method according to claim 40, wherein the cell is a hematopoietic and/or stromal cell.

55. The method according to claim 54, wherein the hematopoietic and/or stromal cell is a bone marrow progenitor cell.

56. The method according to claim 54, wherein the hematopoietic and/or stromal cell is a bone marrow precursor cell.

57. The method according to claim 54, wherein the hematopoietic and/or stromal cell is a mature bone marrow cell.

58. The method according to claim 54, wherein the hematopoietic and/or stromal cell is a stem cell.

59. The method according to claim 54, wherein the clock controlled gene is GM-CSF.

60. The method according to claim 59, wherein said manipulating activates GM-CSF expression.

61. The method according to claim 59, wherein said manipulating deactivates GM-CSF expression.

62. The method according to claim 59, wherein said manipulating alters GM-CSF expression to enhance the immune system and/or influence cell differentiation and/or potency.

63. The method according to claim 59, wherein said manipulating alters GM-CSF expression to treat diseases mediated by GM-CSF or its deficiency.

64. The method according to claim 54, wherein the clock controlled gene is IL-12 or IL-16.

65. The method according to claim 64, wherein said manipulating activates IL-12 or IL-16 expression.

66. The method according to claim 64, wherein said manipulating deactivates IL-12 or IL-16 expression.

67. The method according to claim 64, wherein said manipulating alters IL-12 or IL-16 expression to enhance the immune system and/or influence cell differentiation and/or potency.

68. The method according to claim 64, wherein said manipulating alters IL-12 expression to treat diseases mediated by IL-12 or its deficiency or IL-16 or its deficiency.

69. The method according to claim 54, wherein the clock controlled gene is LATS2.

70. The method according to claim 69, wherein said manipulating activates LATS2 expression.

71. The method according to claim 69, wherein said manipulating deactivates LATS2 expression.

72. The method according to claim 69, wherein said manipulating alters LATS2 expression for treating cancers, leukemias, or other proliferative or malignant diseases.

73. The method according to claim 69, wherein LATS2 is LATS2b.

74. The method according to claim 69, wherein LATS2 is LATS2c.

75. The method according to claim 54, wherein the clock controlled gene is CNTFR.

76. The method according to claim 75, wherein said manipulating activates CNTFR expression.

77. The method according to claim 75, wherein said manipulating deactivates CNTFR expression.

78. The method according to claim 75, wherein said manipulating alters CNTFR expression to affect survival, expansion or differentiation of neuronal cells or stem cells.

79. The method according to claim 54, wherein the clock controlled gene is BMP-2 or BMP-4.

80. The method according to claim 79, wherein said manipulating activates BMP-2 or BMP-4 expression.

81. The method according to claim 79, wherein said manipulating deactivates BMP-2 or BMP-4 expression.

82. The method according to claim 79, wherein said manipulating alters BMP-2 or BMP-4 expression to affect differentiation or maturation to bone cell-like or tooth cell-like cells.

83. The method according to claim 54, wherein the clock controlled gene is TERT.

84. The method according to claim 83, wherein said manipulating activates TERT expression.

85. The method according to claim 83, wherein said manipulating deactivates TERT expression.

86. The method according to claim 83, wherein said manipulating alters TERT expression to increase the number of potential doublings of a cell.

87. The method according to claim 83, wherein said manipulating alters TERT expression to decrease the number of potential doublings of a cell.

88. The method according to claim 83, wherein said cell is a cancer cell, a stem cell, or a lymphocyte.

89. The method according to claim 54, wherein the clock controlled gene is TGF-β1, TGF-β2, or TGF-β3.

90. The method according to claim 89, wherein said manipulating activates TGF-β1, TGF-β2, or TGF-β3 expression.

91. The method according to claim 89, wherein said manipulating deactivates TGF-β1, TGF-β2, or TGF-β3 expression.

92. The method according to claim 89, wherein said manipulating alters TGF-β1, TGF-β2, or TGF-β3 expression to affect cell survival, proliferation, differentiation, or induce apoptosis.

93. The method according to claim 54, wherein the clock controlled gene is Piwi-like-1.

94. The method according to claim 93, wherein said manipulating activates Piwi-like-1 expression.

95. The method according to claim 93, wherein said manipulating deactivates Piwi-like-1 expression.

96. The method according to claim 93, wherein said manipulating alters Piwi-like-1 expression to affect cell division.

97. The method according to claim 93, wherein the cell is a stem cell.

98. The method according to claim 54, wherein the clock controlled gene is CEBP-α.

99. The method according to claim 98, wherein said manipulating activates CEBP-α expression.

100. The method according to claim 98, wherein said manipulating deactivates CEBP-α expression.

101. The method according to claim 98, wherein said manipulating alters CEBP-α expression to affect lineage commitment.

102. The method according to claim 54, wherein the clock controlled gene is DMP-1.

103. The method according to claim 102, wherein said manipulating activates DMP-1 expression.

104. The method according to claim 102, wherein said manipulating deactivates DMP-1 expression.

105. The method according to claim 102, wherein said manipulating alters DMP-1 expression to affect differentiation to tooth cell-like cells.

106. The method according to claim 54, wherein the clock controlled gene is OASIS.

107. The method according to claim 106, wherein said manipulating activates OASIS expression.

108. The method according to claim 106, wherein said manipulating deactivates OASIS expression.

109. The method according to claim 106, wherein said manipulating alters OASIS expression to affect osteoblast differentiation and/or maturation.

110. The method according to claim 54, wherein the clock controlled gene is lim-homeobox-2 or homeobox-4.

111. The method according to claim 110, wherein said manipulating activates lim-homeobox-2 or homeobox-4 expression.

112. The method according to claim 110, wherein said manipulating deactivates lim-homeobox-2 or homeobox-4 expression.

113. The method according to claim 110, wherein said manipulating alters lim-homeobox-2 or homeobox-4 expression to generate, expand or maintain hematopoictic stem cells.

114. The method according to claim 54, wherein the clock controlled gene is Pax5.

115. The method according to claim 114, wherein said manipulating activates Pax5 expression.

116. The method according to claim 114, wherein said manipulating deactivates Pax5 expression.

117. The method according to claim 114, wherein said manipulating alters Pax5 expression to affect lymphocyte development, neuronal cell development, or spermatogenesis.
